(19) United States
(12) Patent Application Publication
(10) Pub. No.: US 2024/0189304 A1
(43) Pub. Date: Jun. 13, 2024

(54) BET PROTEIN INHIBITORS AND USE THEREOF

(71) Applicants: The Regents of the University of California, Oakland, CA (US); Ohio State Innovation Foundation, Columbus, OH (US)

(72) Inventors: Allen C. Gao, Davis, CA (US); Pui-Kai Li, Columbus, OH (US); Mark Foster, Columbus, OH (US); Ross Larue, Columbus, OH (US); Cameron M. Armstrong, Davis, CA (US); Wei Lou, Davis, CA (US); Shu Ning, Davis, CA (US); Enming Xing, Columbus, OH (US)

(73) Assignees: The Regents of the University of California, Oakland, CA (US); Ohio State Innovation Foundation, Columbus, OH (US); The United States Government as represented by the Department of Veterans Affairs, Washington, DC (US)

(21) Appl. No.: 18/548,844

(22) PCT Filed: Mar. 2, 2022
(86) PCT No.: PCT/US2022/018535

§ 371 Date: Sep. 1, 2023

Related U.S. Application Data
(60) Provisional application No. 63/155,854, filed on Mar. 3, 2021.

(51) Int. Cl. A61K 31/496; A61K 31/4155; A61K 31/4166; A61K 31/4439; A61K 31/454; A61K 31/58; A61P 35/00; C07D 401/12; C07D 405/12

(52) U.S. Cl. CPC A61K 31/496; A61K 31/4155; A61K 31/4166; A61K 31/4439; A61K 31/454; A61K 31/58; A61P 35/00; C07D 401/12; C07D 405/12

(57) ABSTRACT

Methods for the treatment of condition associated with bromodomain and extraterminal domain (BET) protein activity are disclosed. The methods include administering a therapeutically effective amount of a piperazine BET protein inhibitor or a piperidine BET protein inhibitor to a subject in need thereof. Piperazine BET protein inhibitors, piperidine BET protein inhibitors, and pharmaceutical compositions comprising the BET proteins inhibitors are also described.

### CROSS-REFERENCES TO RELATED APPLICATIONS

The present application claims priority to U.S. Provisional Pat. Appl. No. 63/155,854, filed on Mar. 3, 2021, which application is incorporated herein by reference in its entirety.

### STATEMENT AS TO RIGHTS TO INVENTIONS MADE UNDER FEDERALLY SPONSORED RESEARCH AND DEVELOPMENT

This invention was made with government support under Grant No. CA225836 awarded by the National Institutes of Health (NIH). The government has certain rights in the invention.

### BACKGROUND OF THE INVENTION

The bromodomain and extraterminal domain (BET) family of proteins (Brd2, 3, 4, and T), as well as the extended family (Brd1, 7, 8 and 9), are characterized by dual N-terminal bromodomains which bind acetylated H3 and H4 tails on chromatin. BET proteins are distinguished from the extended family by a C-terminal extra-terminal (ET) domain and SEED domain, which contains glutamic and aspartic acid residues interspersed between polyserine residues. Owing to the established role of BET proteins in cell cycle regulation, epigenetic sensing, and a range of disease states, there is considerable interest in the therapeutic potential of targeting BET proteins with small molecule inhibitors. Current methodology relies on acetylation mimics which block the bromodomains from binding chromatin. However, this limits the therapeutic potential as these inhibitors lack selectivity for only the BET proteins and instead targets any proteins containing bromodomains. Other proteins containing bromodomains include, for example, histone-lysine N-methyltransferase protein ASHIL, histone acetyltransferase p300 (EP300), P300/CBP-associated factor (PCAF) and the extended BET family. Perturbation of these other proteins by bromodomain-based inhibitors could lead to off target or detrimental side effects.

### BRIEF SUMMARY OF THE INVENTION

Provided herein are methods for the treatment of conditions associated with BET protein activity, such as inflammation, cancer, cardiovascular disease, and viral infection. The methods include administering a therapeutically effective amount of a diazinane BET protein inhibitor (e.g., a piperazine BET protein inhibitor or a 1,3-diazinane BET protein inhibitor) or a piperidine BET protein inhibitor to a subject in need thereof. In some embodiments, the piperazine BET protein inhibitor is a compound according to Formula I as described herein. In some embodiments, the piperidine BET protein inhibitor is a compound according to Formula II as described herein.

Also provided herein are piperazine BET protein inhibitors according to Formula I:

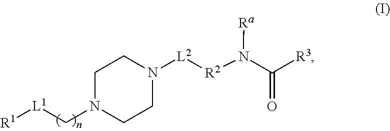

and pharmaceutically acceptable salts thereof,
wherein:
R¹ is selected from the group consisting of C₆₋₁₄ aryl and 5- to 10-membered heteroaryl, each of which is optionally substituted with one more R^{1a},
each R^{1a} is independently selected from the group consisting of halogen, —CN, —NO₂, —NHR^{b}, —N₃, —OH, —SH, —SO₃H, C₁₋₈ alkyl, C₁₋₈ alkoxy, C₂₋₈ alkenyl, C₂₋₈ alkynyl, —COOR^{b}, —C(O)NHR^{b}, and —C(O)R^{c},
—L¹— is selected from the group consisting of —O—, —S—, and —NR^{a}—;
—L²— is selected from the group consisting of —C(O)— and —SO₂—;
—R²— is selected from the group consisting of phenylene, pyrrol-diyl, furan-diyl, and thiophen-diyl;
R³ is selected from the group consisting of C₃₋₈ cycloalkyl, 3- to 10-membered heterocyclyl, C₆₋₁₄ aryl, and 5- to 10-membered heteroaryl, each of which is optionally substituted with one or more R^{3a},
each R^{3a} is independently selected from the group consisting of halogen, —CN, —NO₂, —NHR^{b}, —N₃, —OH, —SH, —SO₃H, C₁₋₈ alkyl, C₁₋₈ alkoxy, C₂₋₈ alkenyl, C₂₋₈ alkynyl, —COOR^{b}, —C(O)NHR^{b}, and —C(O)R^{c};
alternatively, the grouping —C(O)R³ is an α-aminoacyl moiety;
subscript n is 1, 2, or 3,
each R^{a} and each R^{b} is independently selected from the group consisting of H and C₁₋₄ alkyl; and
each R^{c} is independently C₁₋₄ alkyl.

Also provided herein are piperidine BET protein inhibitors according to Formula II:

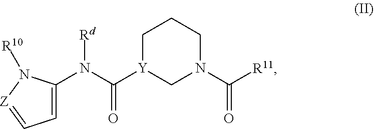

and pharmaceutically acceptable salts thereof,
wherein:
Y is CH or N;
Z is CH, N, or O;
R¹⁰ is selected from the group consisting of H, C₁₋₈ alkyl, C₃₋₈ cycloalkyl, C₂₋₈ alkenyl, and C₂₋₈ alkynyl, each of which is optionally substituted with one or more R^{10a};
each R^{10a} is independently selected from the group consisting of halogen, —CN, —NO₂, —NHR^{e}, —N₃, —OH, —SH, —SO₃H, C₁₋₈ alkyl, C₁₋₈ alkoxy, C₂₋₈ alkenyl, C₂₋₈ alkynyl, —COOR^{e}, —C(O)NHR^{e}, and —C(O)R^{f};
R¹¹ is selected from the group consisting of C₆₋₁₄ aryl, and 5- to 10-membered heteroaryl, each of which is optionally substituted with one or more R^{11a};
each R^{11a} is independently selected from the group consisting of halogen, —CN, —NO₂, —NHR^{e}, —N₃, —OH, —SH, —SO₃H, C₁₋₈ alkyl, C₁₋₈ alkoxy, C₂₋₈ alkenyl, C₂₋₈ alkynyl, —COOR^{e}, —C(O)NHR^{e}, and —C(O)R^{f};
R^{d} and each R^{e} is selected from the group consisting of H and C₁₋₄ alkyl; and
each R^{f} is independently C₁₋₄ alkyl.

### BRIEF DESCRIPTION OF THE DRAWINGS

FIG. 1 shows the inhibition of enzalutamide- and darolutamide-resistant cancer cell

growth by piperazine BET protein inhibitor 10.

FIG. 2 shows the inhibition of C4-2B MDVR (enzalutamide resistant) cell growth by piperazine BET protein inhibitor 10 in a dose-dependent manner.

FIG. 3A shows the inhibition of BRD4, AR-FL, and cMYC expression by piperazine BET protein inhibitor 10 in C4-2B MDVR cells.

FIG. 3B shows the inhibition of cMYC expression by piperazine BET protein inhibitors 10-13 (2.5 μM) in C4-2B MDVR cells.

FIG. 4 shows that piperazine BET protein inhibitor 10 synergizes with antiandrogen drugs to inhibit the growth of cancer cells.

FIG. 5 shows the inhibition of C4-2B and C4-2B MDVR cell growth by piperidine BET protein inhibitor 2 in a dose-dependent manner.

FIG. 6A shows that piperidine BET protein inhibitor 2 synergizes with enzalutamide in inhibiting the growth of C4-2B MDVR cells.

FIG. 6B shows cell numbers counted at 5 days for a C4-2B MDVR cell culture of FIG. 6A.

FIG. 7A shows fluorescence micrographs of LuCaP 35CR organoids treated with piperazine BET protein inhibitor 10 at concentrations as indicated for 7 days.

FIG. 7B shows organoid viability for organoids treated with BET protein inhibitor 10 alone.

FIG. 7C shows fluorescence micrographs of LuCaP 35CR organoids treated with piperazine BET protein inhibitor 10, at concentrations as indicated, in combination with enzalutamide 20 μM for 7 days.

FIG. 7D shows organoid viability for organoids treated with BET protein inhibitor 10 and enzalutamide.

FIG. 7E shows the computational assessment of synergistic effects of piperazine BET protein inhibitor 10 and enzalutamide. The coefficient of drug interaction (CDI) of 20 μM enzalutamide with 0.5 μM BET protein inhibitor combination treatment were 0.97 and 0.91 respectively in LuCaP35CR organoids, evidencing a synergistic effect (CDI=0.4222, <1).

FIG. 8 shows that piperazine BET inhibitors 10-15 inhibit prostate cancer cell growth.

FIG. 9 shows that piperazine BET inhibitors synergize with enzalutamide in inhibiting the growth of prostate cancer cells.

FIG. 10 shows that piperazine BET inhibitor 10 inhibits the growth of prostate cancer tumors in vivo.

### DETAILED DESCRIPTION OF THE INVENTION

The present disclosure provide inhibitors of BET proteins and methods for the treatment of conditions related to BET protein activity. In contrast to known BET inhibitors that target the bromodomain of proteins such as BRD2, BRD3, and BRD4, compounds according to the present disclosure have been designed to bind to the extraterminal domain of target BET proteins.

### I. Definitions

As used herein the terms “BET protein” and “bromodomain and extraterminal domain protein” refer to a family of proteins, including BRD2, BRD3, BRD4, and BRDT, that are understood to function as major transcriptional regulators. BET proteins contain two tandem bromodomains, sharing a conserved left-handed four helix bundle. Inter-helical loop regions form an acetyl-lysine binding pocket located at one end of the helix bundle. The BET family is conserved across a wide variety of species; the family includes Saccharomyces cerevisiae bromodomain factor 1 (bdf1) and bromodomain factor 2 (bdf2), Drosophila melanogaster female sterile homeotic protein [fs(1)h], and mammalian BRD2, BRD3, BRD4, and testes/oocyte-specific BRDT/BRD6.

As used herein, the term “alkyl,” by itself or as part of another substituent, refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆, and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted. Unless otherwise specified, “substituted alkyl” groups may be substituted with one or more groups selected from halo, hydroxy, amino, alkylamino, amido, acyl, nitro, cyano, and alkoxy.

As used herein, the term “alkoxy,” by itself or as part of another substituent, refers to a group having the formula —OR, wherein R is alkyl as described above.

As used herein, the term “alkenyl,” by itself or as part of another substituent, refers to a straight chain or branched hydrocarbon having at least 2 carbon atoms and at least one double bond. Alkenyl can include any number of carbons, such as C₂, C₂₋₃, C₂₋₄, C₂₋₅, C_{2-6,} C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted. Unless otherwise specified, “substituted alkenyl” groups may be substituted with one or more moieties selected from halo, hydroxy, amino, alkylamino, alkoxy, carboxy, amido, nitro, oxo, and cyano.

As used herein, the term “alkynyl,” by itself or as part of another substituent, refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be substituted or unsubstituted. Unless otherwise specified, “substituted alkynyl” groups may be substituted with one or more moieties selected from halo, hydroxy, amino, alkylamino, alkoxy, carboxy, amido, nitro, oxo, and cyano.

As used herein, the terms “halo” and “halogen” refer to fluorine, chlorine, bromine and iodine.

As used herein, the term “hydroxy” refers to the moiety —OH.

As used herein, the term “Oxo” refers to an oxygen atom that is double-bonded to a compound (i.e., O═).

As used herein, the term “amino” refers to a moiety —NR₂, wherein each R group is H or alkyl. An amino moiety can be ionized to form the corresponding ammonium cation. “Alkylamino” refers to an amino moiety wherein at least one of the R groups is alkyl.

As used herein, the term “α-aminoacyl” refers to a moiety containing a carbonyl group adjacent to a carbon bonded to an amine. For example, the α-aminoacyl moiety may have the formula —C(O)C(NR₂)R′R″, wherein each R is independently hydrogen or alkyl, and wherein R′ and R″ are independently hydrogen, alkyl, alkynyl, aryl, heteroaryl, cycloalkyl, or heterocyclyl, each of which is optionally substituted with one or more substituents selected from halo, hydroxy, amino, alkylamino, amido, acyl, nitro, cyano, and alkoxy.

As used herein, the term “aryl,” by itself or as part of another substituent, refers to an aromatic ring system having any suitable number of carbon ring atoms and any suitable number of rings. Aryl groups can include any suitable number of carbon ring atoms, such as C₆, C₇, C₈, C₉, C₁₀, C₁₁, C₁₂, C₁₃, C₁₄, C₁₅ or C₁₆, as well as C₆₋₁₀, C₆₋₁₂, or C₆₋₁₄. Aryl groups can be monocyclic, fused to form bicyclic (e.g., benzocyclohexyl) or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted. Unless otherwise specified, “substituted aryl” groups can be substituted with one or more groups selected from halo, hydroxy, amino, alkylamino, amido, acyl, nitro, cyano, and alkoxy.

As used herein, the term “heteroaryl,” by itself or as part of another substituent, refers to a monocyclic or fused bicyclic or tricyclic aromatic ring assembly containing 5 to 16 ring atoms, where from 1 to 5 of the ring atoms are a heteroatom such as N, O or S. Additional heteroatoms can also be useful, including, but not limited to, B, Al, Si and P. The heteroatoms can be oxidized to form moieties such as, but not limited to, —S(O)— and —S(O)2—. Heteroaryl groups can include any number of ring atoms, such as C₅₋₆, C₃₋₈, C₄₋₈, C₅₋₈, C₆₋₈, C₃₋₉, C₃₋₁₀, C₃₋₁₁, or C₃₋₁₂, wherein at least one of the carbon atoms is replaced by a heteroatom. Any suitable number of heteroatoms can be included in the heteroaryl groups, such as 1, 2, 3, 4; or 5, or 1 to 2, 1 to 3, 1 to 4, 1 to 5, 2 to 3, 2 to 4, 2 to 5, 3 to 4, or 3 to 5. For example, heteroaryl groups can be C₅₋₈ heteroaryl, wherein 1 to 4 carbon ring atoms are replaced with heteroatoms; or C₅₋₈ heteroaryl, wherein 1 to 3 carbon ring atoms are replaced with heteroatoms; or C₅₋₆ heteroaryl, wherein 1 to 4 carbon ring atoms are replaced with heteroatoms; or C₅₋₆ heteroaryl, wherein 1 to 3 carbon ring atoms are replaced with heteroatoms. The heteroaryl group can include groups such as pyrrole, pyridine, imidazole, pyrazole, triazole, tetrazole, pyrazine, pyrimidine, pyridazine, triazine (1,2,3, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted. Unless otherwise specified, “substituted heteroaryl” groups can be substituted with one or more groups selected from halo, hydroxy. amino, alkylamino, amido, acyl, nitro, cyano, and alkoxy.

The heteroaryl groups can be linked via any position on the ring. For example, pyrrole includes 1, 2- and 3-pyrrole, pyridine includes 2, 3- and 4-pyridine, imidazole includes 1, 2, 4- and 5-imidazole, pyrazole includes 1, 3, 4- and 5-pyrazole, triazole includes 1, 4- and 5-triazole, tetrazole includes 1- and 5-tetrazole, pyrimidine includes 2, 4, 5- and 6- pyrimidine, pyridazine includes 3- and 4-pyridazine, 1,2,3-triazine includes 4- and 5-triazine, 1,2,4-triazine includes 3, 5- and 6-triazine, 1,3,5-triazine includes 2-triazine, thiophene includes 2- and 3-thiophene, furan includes 2- and 3-furan, thiazole includes 2, 4- and 5-thiazole, isothiazole includes 3, 4- and 5-isothiazole, oxazole includes 2, 4- and 5-oxazole, isoxazole includes 3, 4- and 5-isoxazole, indole includes 1, 2- and 3-indole, isoindole includes 1- and 2-isoindole, quinoline includes 2, 3- and 4-quinoline, isoquinoline includes 1, 3- and 4-isoquinoline, quinazoline includes 2- and 4-quinazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

Some heteroaryl groups include from 5 to 10 ring members and only nitrogen heteroatoms, such as pyrrole, pyridine, imidazole, pyrazole, triazole, pyrazine, pyrimidine, pyridazine, triazine (1,2,3, 1,2,4- and 1,3,5-isomers), indole, isoindole, quinoline, isoquinoline, quinoxaline, quinazoline, phthalazine, and cinnoline. Other heteroaryl groups include from 5 to 10 ring members and only oxygen heteroatoms, such as furan and benzofuran. Some other heteroaryl groups include from 5 to 10 ring members and only sulfur heteroatoms, such as thiophene and benzothiophene. Still other heteroaryl groups include from 5 to 10 ring members and at least two heteroatoms, such as imidazole, pyrazole, triazole, pyrazine, pyrimidine, pyridazine, triazine (1,2,3, 1,2,4- and 1,3,5-isomers), thiazole, isothiazole, oxazole, isoxazole, quinoxaline, quinazoline, phthalazine, and cinnoline.

As used herein, the term “cycloalkyl,” by itself or as part of another substituent, refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted. Unless otherwise specified, “substituted cycloalkyl” groups can be substituted with one or more groups selected from halo, hydroxy, amino, alkylamino, amido, acyl, nitro, cyano, and alkoxy.

As used herein the term “heterocyclyl,” by itself or as part of another substituent, refers to a saturated ring system having from 3 to 12 ring members and from 1 to 4 heteroatoms of N, O and S. Additional heteroatoms can also be useful, including, but not limited to, B, Al, Si and P. The heteroatoms can be oxidized to form moieties such as, but not limited to, —S(O)— and —S(O)₂—. Heterocyclyl groups can include any number of ring atoms, such as, C₃₋₆, C₄₋₆, C₅₋₆, C₃₋₈, C₄₋₈, C₅₋₈, C₆₋₈, C₃₋₉, C₃₋₁₀, C₃₋₁₁, or C₃₋₁₂, wherein at least one of the carbon atoms is replaced by a heteroatom. Any suitable number of carbon ring atoms can be replaced with heteroatoms in the heterocyclyl groups, such as 1, 2, 3, or 4, or 1 to 2, 1 to 3, 1 to 4, 2 to 3, 2 to 4, or 3 to 4. The heterocyclyl group can include groups such as aziridine, azetidine, pyrrolidine, piperidine, azepane, azocane, quinuclidine, pyrazolidine, imidazolidine, piperazine (1,2, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocyclyl groups can also be fused to aromatic or non-aromatic ring systems to form members including, but not limited to, indoline. Heterocyclyl groups can be unsubstituted or substituted. Unless otherwise specified, “substituted heterocyclyl” groups can be substituted with one or more groups selected from halo, hydroxy, amino, oxo (═O), alkylamino, amido, acyl, nitro, cyano, and alkoxy.

The heterocyclyl groups can be linked via any position on the ring. For example, aziridine can be 1- or 2-aziridine, azetidine can be 1- or 2- azetidine, pyrrolidine can be 1, 2- or 3-pyrrolidine, piperidine can be 1, 2, 3- or 4-piperidine, pyrazolidine can be 1, 2, 3, or 4-pyrazolidine, imidazolidine can be 1, 2, 3- or 4-imidazolidine, piperazine can be 1, 2, 3- or 4-piperazine, tetrahydrofuran can be 1- or 2-tetrahydrofuran, oxazolidine can be 2, 3, 4- or 5-oxazolidine, isoxazolidine can be 2, 3, 4- or 5-isoxazolidine, thiazolidine can be 2, 3, 4- or 5-thiazolidine, isothiazolidine can be 2, 3, 4- or 5- isothiazolidine, and morpholine can be 2, 3- or 4-morpholine.

When heterocyclyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxazolidine, thiazolidine, isothiazolidine, morpholine, thiomorpholine, dioxane and dithiane. Heterocyclyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

As used herein, the term “amido” refers to a moiety —NRC(O)R or —C(O)NR₂, wherein each R group is H or alkyl.

As used herein, the term “acyl” refers to the moiety —C(O)R, wherein each R group is alkyl.

As used herein, the term “nitro” refers to the moiety —NO₂.

As used herein, the term “cyano” refers to a carbon atom triple-bonded to a nitrogen atom (i.e., the moiety —C≡N).

As used herein, the term “carboxy” refers to the moiety —C(O)OH.

As used herein, the term “salt” refers to an acid salt or base salt of an active agent such as BET protein inhibitor. Acid salts of basic active agents include mineral acid salts (e.g., salts formed by using hydrochloric acid, hydrobromic acid, phosphoric acid, and the like), organic acid salts (e.g., salts formed using acetic acid, propionic acid, glutamic acid, citric acid, and the like), and quaternary ammonium salts (e.g., salts formed via reaction of an amine with methyl iodide, ethyl iodide, or the like). It is understood that the pharmaceutically acceptable salts are non-toxic.

Acidic active agents may be contacted with bases to provide base salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

The neutral forms of the active agents can be regenerated by contacting the salt with a base or acid and isolating the parent compound in the conventional manner if desired. In some embodiments, the parent form of the compound may differ from various salt forms in certain physical properties, such as solubility in polar solvents, but otherwise the salts forms may be equivalent to the parent form of the compound.

By “pharmaceutically acceptable,” it is meant that the excipient is compatible with the other ingredients of the formulation and is not deleterious to the recipient thereof. As used herein, the term “pharmaceutically acceptable excipient” refers to a substance that aids the administration of an active agent to a subject. Useful pharmaceutical excipients include, but are not limited to, binders, fillers, disintegrants, lubricants, glidants, coatings, sweeteners, flavors and colors.

As used herein, the terms “effective amount” and “therapeutically effective amount”

refer to a dose of a compound such as a BET protein inhibitor or an antiandrogen that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition, 2006, Brunton, Ed., McGraw-Hill; and Remington: The Science and Practice of Pharmacy, 21^{st} Edition, 2005, Hendrickson, Ed., Lippincott, Williams & Wilkins).

As used herein, the term “cancer” is intended to include any member of a class of diseases characterized by the uncontrolled growth of aberrant cells. The term includes all known cancers and neoplastic conditions, whether characterized as malignant, benign, recurrent, soft tissue, or solid, and cancers of all stages and grades including advanced, recurrent, pre- and post-metastatic cancers. Additionally, the term includes androgen-independent, castrate-resistant, castration recurrent, hormone-resistant, drug-resistant, and metastatic castrate-resistant cancers. Examples of different types of cancer include, but are not limited to, prostate cancer (e.g., prostate adenocarcinoma); breast cancers (e.g., triple-negative breast cancer, ductal carcinoma in situ, invasive ductal carcinoma, tubular carcinoma, medullary carcinoma, mucinous carcinoma, papillary carcinoma, cribriform carcinoma, invasive lobular carcinoma, inflammatory breast cancer, lobular carcinoma in situ, Paget's disease, Phyllodes tumors); gynecological cancers (e.g., ovarian, cervical, uterine, vaginal, and vulvar cancers); lung cancers (e.g., non-small cell lung cancer, small cell lung cancer, mesothelioma, carcinoid tumors, lung adenocarcinoma); digestive and gastrointestinal cancers such as gastric cancer (e.g., stomach cancer), colorectal cancer, gastrointestinal stromal tumors (GIST), gastrointestinal carcinoid tumors, colon cancer, rectal cancer, anal cancer, bile duct cancer, small intestine cancer, and esophageal cancer; thyroid cancer; gallbladder cancer; liver cancer; pancreatic cancer; appendix cancer; renal cancer (e.g., renal cell carcinoma); cancer of the central nervous system (e.g., glioblastoma, neuroblastoma); skin cancer (e.g., melanoma); bone and soft tissue sarcomas (e.g., Ewing's sarcoma); lymphomas; choriocarcinomas; urinary cancers (e.g., urothelial bladder cancer); head and neck cancers; and bone marrow and blood cancers (e.g., chronic lymphocytic leukemia, lymphoma). As used herein, a “tumor” comprises one or more cancerous cells.

As used herein, the terms “about” and “around” indicate a close range around a numerical value when used to modify that specific value. If “X” were the value, for example, “about X” or “around X” would indicate a value from 0.9X to 1.1X, e.g., a value from 0.95X to 1.05X, or a value from 0.98X to 1.02X, or a value from 0.99X to 1.01X. Any reference to “about X” or “around X” specifically indicates at least the values X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.1X, and values within this range.

### II. Embodiments of the Invention

The extraterminal (ET) domain of BET proteins (bromodomain and extraterminal domain proteins) associates with a variety of cellular proteins including chromatin-modifying factors, transcription factors, histone modification enzymes, and a number of viral proteins. These interacting partners include jumonji C-domain-containing protein 6 (JMJD6), histone-lysine N-methyltransferase NSD3 (NSD3), glioma tumor suppressor candidate region gene 1 protein (GLTSCR1), ATPase family AAA domain-containing protein 5 (ATAD5), and chromodomain helicase DNA-binding protein 4 (CHD4), as well as viral γ-2 herpesvirus latency-associated nuclear antigen and integrase (IN) from murine leukemia virus (MLV). These ET domain interacting partners highlight the role of the ET domain in a variety of human cancers. For example, JMJD6-Brd4 interactions have been implicated in multiple cancers including oral, breast, lung and colon. NSD3 interactions with Brd4 are important in an aggressive midline carcinoma resulting from a chromosomal translocation that fuses Brd4 with the nuclear protein in testes, as well as cellular interactions of Brd4-NSD3 which have been shown to be essential for acute myeloid leukemia maintenance. The role of MLV in human cancers was first demonstrated in hematopoietic stem cell (HSC) gene-therapy studies which showed that insertion of MLV-based vectors near the LMO-2 and CCND2 proto-oncogenes resulted in leukemic development. This insertional basis of MLV for proto-oncogenes is believed to be due to selective high affinity binding of MLV IN to cellular BET proteins which guides integration to select regions of chromatin. This initial discovery led to functional and structural studies of MLV IN-Brd4 ET interactions which have examined the binding interface and essential interactions in depth.

A 17 amino acid region TWRVQRSQNPLKIRKTR(389′-405′) (SEQ ID NO:1) located in the C-terminus of the C-terminal domain of MLV IN was found to be the minimal binding region for interaction with Brd4 ET (termed the ET binding motif or EBM). This peptide construct effectively out-competed transcription factor NSD3 for binding to full-length Brd4 due to its high binding affinity (˜160 nM). The NMR solution structure of EBM bound to BRD4 ET domain was solved, revealing key interactions including complementary electrostatic interactions between the negatively charged side chains on the conserved DEIDIDF(650′-656′) (SEQ ID NO:2) of the ET domain and the positively charged side chains on the conserved LKIRLTR(399′-405′) (SEQ ID NO:3) of MLV IN and favorable hydrophobic interactions involving residues contributed by helices α1 and α2 and the strand β1 of the ET domain (L^{630,} V634, I652, I654, and F656) and both strands of the EBM (W390′, V392′, L^{399′,} I401′, and L^{403′}).

Subsequent mutational analysis confirmed the essential nature of Brd4 E653R/D655R and V634S/I652S resides for interaction with MLV IN and cellular cofactors including NSD3. These alternating hydrophobic and charged amino acids of MLV IN were identified for exploitation in library screening, drug development, and biological screening. Biological screening confirmed the inhibitory potential of EBM to disrupt BRD4 mediated acute myeloid leukemia AML in Molm-13 cells. These cells were transduced to allow the stable expression of EBM which showed almost 10 fold reduction in cellular proliferation compared to vector control cells seven days post transduction. The LKIRL(399′-403′) (SEQ ID NO:4) motif of EMB retained ˜80% inhibitor potential when compared to the full EBM utilizing a similar setup. A global docking study showed that LKIRL binds preferably to the MLV-IN/ET protein-protein interaction interface even though it doesn't contain all the amino acids of the original beta-sheet. Molecular dynamic simulations of resulting binding poses further support the stability and feasibility of the interaction between LKIRL and ET. Preliminary biological evaluation provided further evidence to the hypothesis that LKIRL showed almost identical inhibitory activity compared to that of the original longer version. As described in more detail below, diazinane- and piperidine-containing compounds for efficacious inhibition of BET proteins have now been discovered. The activity of the compounds is believed to arise, in part, because they share certain structural features of the LKIRL motif.

Provided herein are methods for the treatment of conditions associated with BET protein activity. The methods include administering a therapeutically effective amount of a diazinane BET protein inhibitor (e.g., a piperazine BET protein inhibitor or a 1,3-diazinane BET protein inhibitor) or a piperidine BET protein inhibitor to a subject in need thereof.

In some embodiments, the condition associated with BET protein activity is selected from the group consisting of inflammation, cancer, cardiovascular disease, and a viral infection.

In some embodiments, the disease is a cancer. In some embodiments, the cancer overexpresses a BET protein such as BRD2, BRD3, or BRD4 (e.g., a cancer as set forth by Stathis et al. “BET Proteins as Targets for Anticancer Treatment.” Cancer Discov 2018 (8) (1) 24-36; and references cited therein). The cancer may be, for example, a carcinoma of the breast, prostate, endometrium, or kidney; hepatocellular carcinoma; a bladder cancer; a renal cancer; a gastric cancer; a cervical cancer; a colon cancer; or a lung cancer (e.g., non-small cell lung cancer; NSCLC). In some embodiments, the cancer is an oral cancer, a breast cancer, a prostate cancer, a lung cancer, or a colon cancer. The cancer may be an advanced stage cancer. The cancer may be a metastatic cancer. The cancer may be a drug-resistant cancer (e.g., a hormone drug-resistant cancer or a chemotherapy-resistant cancer).

In some embodiments, the treatment of cancer includes inhibiting growth of cancer cells (e.g., prostate, breast, ovarian, or liver cancer cell), migration of cancer cells, or invasion of cancer cells into tissues and/or organs. The treatment may include ameliorating the symptoms of cancer, reducing tumor size, reducing cancer cell and/or tumor numbers. The treatment may include inducing cancer cell necrosis, pyroptosis, oncosis, apoptosis, autophagy, or other cell death.

In some embodiments, the cancer is a prostate cancer. The cancer may be a castrate-resistant cancer, which is not effectively treated by surgical castration (orchiectomy) or drugs such as luteinizing hormone-releasing hormone (LHRH) agonists, LHRH antagonists, CYP17 inhibitors, and androgen receptor antagonists. Examples of LHRH agonists include, but are not limited to, leuprolide, goserelin, triptorelin, histrelin, and the like. Examples of LHRH antagonists include, but are not limited to, degarelix, relugolix, and the like. Examples of CYP17 inhibitors include, but are not limited to, abiraterone, ketoconazole orteronel, viamet, galeterone, 1-(2-chloro-pyridin-4-yl)-3-(4-methyl-pyridin-3-yl) 2-imidazolidinone (CFG-290), (1S)-1-(6,7-dimethoxy-2-naphthyl)-1-(1H-imidazol-4-yl)-2-methylpropan-1-ol (TAK-700), 3β-hydroxy-17-(1H-benzimidazol-1-yl)androsta-5,16-diene (TOK-001), and the like. Examples of androgen receptor antagonists include, but are not limited to, flutamide, bicalutamide, nilutamide, enzalutamide, apalutamide, darolutamide, and the like. Resistance to drugs such as androgen receptor antagonists may be due, in whole or in part, to expression of AR splice variants including AR-V1, AR-V3, AR-V7, AR-V9, and AR-V12.

In some embodiments, treating cancer (e.g., prostate cancer), includes enhancing the therapeutic effects of an antiandrogen drug (e.g., a non-steroidal androgen receptor antagonist or a CYP17A1 inhibitor). As used herein, the terms “antiandrogen” and “antiandrogen drug” refer to compounds that alter the androgen pathway by blocking the androgen receptors, competing for binding sites on the cell's surface, or affecting or mediating androgen production. In some embodiments, treatment comprises enhancing the therapeutic effects of enzalutamide, apalutamide, bicalutamide, or abiraterone. The enhancement can be synergistic or additive. In some embodiments, treatment comprises reversing, reducing, or decreasing the resistance of cancer cells (e.g., prostate cancer cells, breast cancer cells, ovarian cancer cells, or liver cancer cells) to antiandrogen drugs. In some embodiments, the treatment comprises resensitizing cancer cells (e.g., prostate cancer cells or breast cancer cells) to antiandrogen drugs.

In some embodiments, the condition is a viral infection. The infection may be caused by DNA viruses, such as Herpesviridae (e.g., psuedorabies virus (PRV), herpes simplex virus 1 (HSV1)), Papillomaviridae (e.g., human papillomavirus HPV), and Poxviridae (e.g., ectromelia virus (ECTV)), as well as RNA viruses such as Orthomyxoviridae (e.g., influenza A/H1N₁), Retroviridae (e.g., murine leukemia virus (MLV), human immunodeficiency virus (HIV), Rhabdoviridae (e.g., vesicular stomatitis virus (VSV)), Arteriviridae (e.g., porcine reproductive and respiratory syndrome virus (PRRSV)), and Paramyxoviridae (e.g., Newcastle disease virus (NDV)).

In some embodiments, the condition is a cardiovascular disease. The BET protein inhibitor may be used, for example, to treat pulmonary arterial hypertension, heart failure, atherosclerosis, hypertension, or a combination thereof.

### A. Piperazine BET Protein Inhibitors

In some embodiments, a piperazine BET protein inhibitor is administered to the subject. The piperazine BET protein inhibitor may be, for example, a compound according to Formula I:

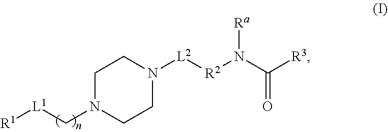

or a pharmaceutically acceptable salt thereof,
wherein:
R¹ is selected from the group consisting of C₆₋₁₄ aryl and 5- to 10-membered heteroaryl, each of which is optionally substituted with one more R^{1a},
each R^{1a} is independently selected from the group consisting of halogen, —CN, —NO₂, —NHR^{b}, —N₃, —OH, —SH, —SO₃H, C₁₋₈ alkyl, C₁₋₈ alkoxy, C₂₋₈ alkenyl, C₂₋₈ alkynyl, —COOR^{b}, —C(O)NHR^{b}, and —C(O)R^{c},
—L¹— is selected from the group consisting of —O—, —S—, and —NR^{a}—;
—L²— is selected from the group consisting of —C(O)— and —SO₂—;
—R²— is selected from the group consisting of phenylene, pyrrol-diyl, furan-diyl, and thiophen-diyl;
R³ is selected from the group consisting of C₃₋₈ cycloalkyl, 3- to 10-membered heterocyclyl, aryl, and 5- to 10-membered heteroaryl, each of which is optionally substituted with one or more R^{3a},
each R^{3a} is independently selected from the group consisting of halogen, —CN, —NO₂, —NHR^{b}, —N₃, —OH, —SH, —SO₃H, C₁₋₈ alkyl, C₁₋₈ alkoxy, C₂₋₈ alkenyl, C₂₋₈ alkynyl, —COOR^{b}, —C(O)NHR^{b}, and —C(O)R^{c};
alternatively, the grouping —C(O)R³ is an α-aminoacyl moiety;
subscript n is 1, 2, or 3,
each R^{a} and each R^{b} are independently selected from the group consisting of H and C₁₋₄ alkyl; and
each R^{c} is independently C₁₋₄ alkyl.

In some embodiments, R¹ is phenyl which is substituted with one or more R^{1a}. In some embodiments, each R^{1a} is independently halogen. In some embodiments, R¹ is 4-chlorophenyl.

In some embodiments, —L¹— is —O—.

In some embodiments, —L²— is —C(O)—. In some embodiments, R² phenylene (e.g., phen-1,4-diyl or phen-1,5-diyl). In some embodiments, R² is phen-1,5-diyl.

In some embodiments, R³ is 3- to 10-membered heterocyclyl or 5- to 10-membered heteroaryl. R³ may be, for example, tetrahydrofuranyl, pyrrolidinyl, imidazolidinyl, pyrazolidinyl, oxazolidinyl, isoxazolidinyl, thiazolidinyl, isothiazolidnyl, morpholinyl, piperidinyl, piperazinyl, furanyl, pyrrolyl, pyridinyl, imidazolyl, pyrazolyl, triazolyl, tetrazolyl, pyrazinyl, triazinyl, indolyl, isoindolyl, or quinolinyl. In some embodiments, R³ is 5- or 6-membered heterocyclyl or 5- or 6-membered heteroaryl containing one oxygen atom, one nitrogen atom, on sulfur atom, one oxygen atom and one nitrogen atom, one sulfur atom and one nitrogen atom, or two nitrogen atoms. In some embodiments, R³ is furanyl or tetrahydrofuranyl.

In some embodiments, the grouping —C(O)R³ is an α-aminoacyl moiety. For example, R³ in the α-aminoacyl moiety may be —C(NR₂)R′R″, wherein each R is hydrogen or an amine protecting group, R′is hydrogen, and R″ is an amino acid sidechain. R′ may represent, for example, the side chain of a naturally occurring amino acid (e.g., an alanine side chain, an arginine side chain, an asparagine side chain, an aspartic acid side chain, a cysteine side chain, a glutamine side chain, a glutamic acid side chain, a glycine side chain, a histidine side chain, an isoleucine side chain, a leucine side chain, a lysine side chain, a methionine side chain, a phenylalanine side chain, a proline side chain, a selenocysteine side chain, a serine side chain, a threonine side chain, a tryptophan side chain, a tyrosine side chain, or a valine side chain) or the side chain of a non-naturally occurring amino acid (e.g., an azidohomoalanine side chain, a propargylglycine side chain, a p-acetylphenylalanine side chain, or the like).

In some embodiments, the BET protein inhibitor is selected from compounds 10-15 as shown below, and pharmaceutically acceptable salts thereof.

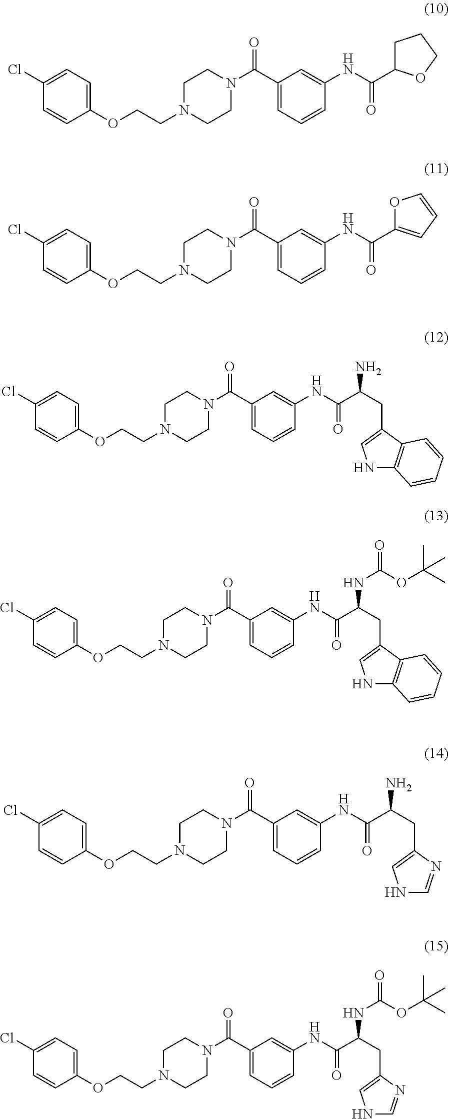 [SMILES: O=C(Nc1cccc(C(=O)N2CCN(CCOc3ccc(Cl)cc3)CC2)c1)C1CCCO1 ; O=C(Nc1cccc(C(=O)N2CCN(CCOc3ccc(Cl)cc3)CC2)c1)c1ccco1 ; N[C@@H](Cc1c[nH]c2ccccc12)C(=O)Nc1cccc(C(=O)N2CCN(CCOc3ccc(Cl)cc3)CC2)c1 ; CC(C)(C)OC(=O)N[C@@H](Cc1c[nH]c2ccccc12)C(=O)Nc1cccc(C(=O)N2CCN(CCOc3ccc(Cl)cc3)CC2)c1 ; N[C@@H](Cc1c[nH]cn1)C(=O)Nc1cccc(C(=O)N2CCN(CCOc3ccc(Cl)cc3)CC2)c1 ; CC(C)(C)OC(=O)N[C@@H](Cc1c[nH]cn1)C(=O)Nc1cccc(C(=O)N2CCN(CCOc3ccc(Cl)cc3)CC2)c1]

In some embodiments, the BET protein inhibitor is piperazine compound 10:

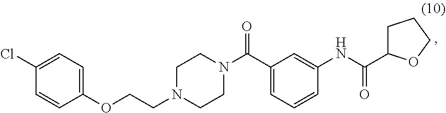 [SMILES: O=C(Nc1cccc(C(=O)N2CCN(CCOc3ccc(Cl)cc3)CC2)c1)C1CCCO1]

or a pharmaceutically acceptable salt thereof.

Also provided herein are compounds according to Formula I:

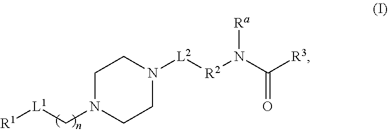

or a pharmaceutically acceptable salt thereof,
wherein:
R¹ is selected from the group consisting of C₆₋₁₄ aryl and 5- to 10-membered heteroaryl, each of which is optionally substituted with one more R^{1a},
each R^{1a} is independently selected from the group consisting of halogen, —CN, —NO₂, —NHR^{b}, —N₃, —OH, —SH, —SO₃H, C₁₋₈ alkyl, C₁₋₈ alkoxy, C₂₋₈ alkenyl, C₂₋₈ alkynyl, —COOR^{b}, —C(O)NHR^{b}, and —C(O)R^{c},
—L¹— is selected from the group consisting of —O—, —S—, and —NR^{a}—;
—L²— is selected from the group consisting of —C(O)— and —SO₂—;
—R²— is selected from the group consisting of phenylene, pyrrol-diyl, furan-diyl, and thiophen-diyl;
R³ is selected from the group consisting of C₃₋₈ cycloalkyl, 3- to 10-membered heterocyclyl, C₆₋₁₄ aryl, and 5- to 10-membered heteroaryl, each of which is optionally substituted with one or more R^{3a},
each R^{3a} is independently selected from the group consisting of halogen, —CN, —NO₂, —NHR^{b}, —N₃, —OH, —SH, —SO₃H, C₁₋₈ alkyl, C₁₋₈ alkoxy, C₂₋₈ alkenyl, C₂₋₈ alkynyl, —COOR^{b}, —C(O)NHR^{b}, and —C(O)R^{c};
alternatively, the grouping —C(O)R³ is an α-aminoacyl moiety (e.g., histidyl, tyrosyl, phenylalanyl, tryptophanyl, or the like),
subscript n is 1, 2, or 3,
each R^{a} and each R^{b} is independently selected from the group consisting of H and C₁₋₄ alkyl; and
each R^{c} is independently C₁₋₄ alkyl;
provided that:
R³ is substituted with at least one R^{3a} when R¹ is unsubstituted phenyl, chlorophenyl, or methoxyphenyl, —L¹— is —O—, —L²— is —C(O)—, R² is phen-1,5-diyl, and R³ is cyclopropyl, tetrahydrofuranyl, or thiophenyl;
R³ is substituted with at least one R^{3a} when R¹ is unsubstituted phenyl or chlorophenyl, —L¹— is —O— or —S—, —L²— is —C(O)—, R² is thiophen-2,4-diyl, and R³ is cyclopropyl or furanyl;
R¹ is substituted with at least one R^{1a} when R¹ is phenyl, —L¹— is —S—, —L²— is —C(O)—, R² is phen-2,6-diyl, and R³ is fluorophenyl, tetrahydrofuranyl, or cyclopropyl.

Piperazine compounds according to Formula I may be synthesized as summarized, for example, in Scheme 1. Protected piperazine (i) may be alkylated with substituted alkane (ii) to form alkylated piperazine (iii), prior to deprotection and formation of dialkylated piperazine (v). Deprotection and acylation with a carboxylic acid R³C(O)OH or an activated derivative (vi) provides the piperazine compound according to Formula I.

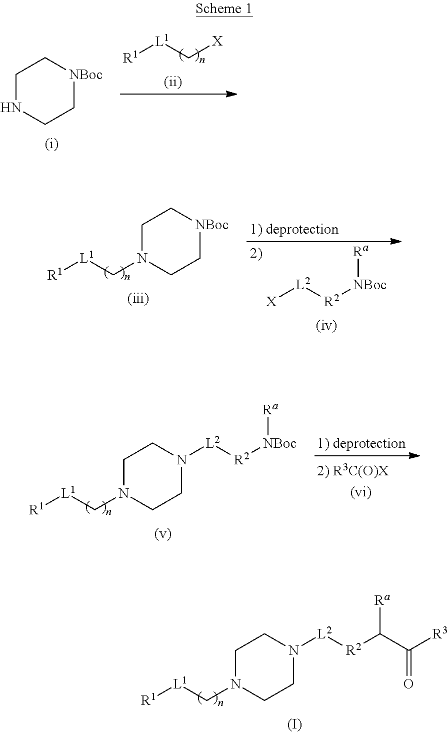

B. Piperidine and 1,3-diazinane BET Protein Inhibitors

In some embodiments, a piperidine or 1,3-diazinane BET protein inhibitor is administered to the subject. The piperidine or 1,3-diazinane BET protein inhibitor may be, for example, a compound according to Formula II:

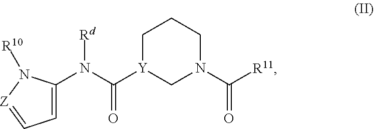

or a pharmaceutically acceptable salt thereof,
wherein:
Y is CH or N;
Z is CH, N, or O;
R¹⁰ is selected from the group consisting of H, C₁₋₈ alkyl, C₃₋₈ cycloalkyl, C₂₋₈ alkenyl, and C₂₋₈ alkynyl, each of which is optionally substituted with one or more R^{10a};
each R^{10a} is independently selected from the group consisting of halogen, —CN, —NO₂, —NHR^{e}, —N₃, —OH, —SH, —SO₃H, C₁₋₈ alkyl, C₁₋₈ alkoxy, C₂₋₈ alkenyl, C₂₋₈ alkynyl, —COOR^{e}, —C(O)NHR^{e}, and —C(O)R^{f};
R¹¹ is selected from the group consisting of C₆₋₁₄ aryl, and 5- to 10-membered heteroaryl, each of which is optionally substituted with one or more R^{11a};
each R^{11a} is independently selected from the group consisting of halogen, —CN, —NO₂, —NHR^{e}, —N₃, —OH, —SH, —SO₃H, C₁₋₈ alkyl, C₁₋₈ alkoxy, C₂₋₈ alkenyl, C₂₋₈ alkynyl, —COOR^{e}, —C(O)NHR^{e}, and —C(O)R^{f};
R^{d} and each R^{e} is selected from the group consisting of H and C₁₋₄ alkyl; and
each R^{f} is independently C₁₋₄ alkyl.

In some embodiments, the BET protein inhibitor is a piperidine compound according to Formula IIa:

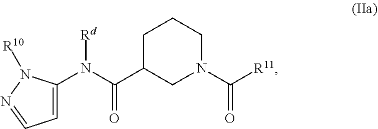

or a pharmaceutically acceptable salt thereof.

In some embodiments, R¹⁰ is C₁₋₈ alkyl in the compound of Formula II or Formula IIa.

In some embodiments, R¹¹ is phenyl which is substituted with one or two R^{11a}. In some embodiments, each R^{11a} is independently halogen in the compound of Formula II or Formula IIa.

In some embodiments, R^{d} is hydrogen in the compound of Formula II or Formula IIa.

In some embodiments, R³ is 3- to 10-membered heterocyclyl in the compound of Formula II or Formula IIa.

In some embodiments, the BET protein inhibitor is piperidine compound 2:

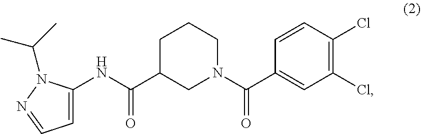 [SMILES: CC(C)n1nccc1NC(=O)C1CCCN(C(=O)c2ccc(Cl)c(Cl)c2)C1]

or a pharmaceutically acceptable salt thereof.

Also provided herein are compounds according to Formula II:

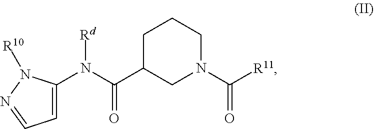

or a pharmaceutically acceptable salt thereof,
wherein:
R¹⁰ is selected from the group consisting of H, C₁₋₈ alkyl, C₃₋₈ cycloalkyl, C₂₋₈ alkenyl, and C₂₋₈ alkynyl, each of which is optionally substituted with one or more R^{10a};
each R^{10a} is independently selected from the group consisting of halogen, —CN, —NO₂, —NHR^{e}, —N₃, —OH, —SH, —SO₃H, C₁₋₈ alkyl, C₁₋₈ alkoxy, C₂₋₈ alkenyl, C₂₋₈ alkynyl, —COOR^{e}, —C(O)NHR^{e}, and —C(O)R^{f};
R¹¹ is selected from the group consisting of C₆₋₁₄ aryl, and 5- to 10-membered heteroaryl, each of which is optionally substituted with one or more R^{11a}.
each R^{11a} is independently selected from the group consisting of halogen, —CN, —NO₂, —NHR^{e}, —N₃, —OH, —SH, —SO₃H, C₁₋₈ alkyl, C₁₋₈ alkoxy, C₂₋₈ alkenyl, C₂₋₈ alkynyl, —COOR^{e}, —C(O)NHR^{e}, and —C(O)R^{f};
R^{d} and each R^{c} is selected from the group consisting of H and C₁₋₄ alkyl; and
each R^{f} is independently C₁₋₄ alkyl;
provided that
R¹¹ is substituted with at least one R^{11a} selected from the group consisting of —CN, —NO₂, —NHR^{e}, —N₃, —OH, —SH, —SO₃H, C₁₋₈ alkoxy, C₂₋₈ alkenyl, C₂₋₈ alkynyl, —COOR^{e}, —C(O)NHR^{e}, and —C(O)R^{f}when R¹¹ is phenyl, Y is CH, Z is N, and R¹⁰ is unsubstituted C₁₋₈ alkyl or unsubstituted C₃₋₈ cycloalkyl; and
R¹¹ is substituted with at least on Rlla when R¹¹ is furan-2-yl or thiophen-2-yl, Y is CH, Z is N, and R¹⁰ is isopropyl, sec-butyl, cyclopentyl, or cyclohexyl.

Piperidine and 1,3-diazinane compounds according to Formula II and Formula IIa may be synthesized as summarized, for example, in Scheme 2. Protected piperidine/diazinane carboxylate (xi) may be coupled with aminoheterocyle (xii) to form amidated piperidine/diazinane (xiii), prior to optional alkylation of the heterocyclic moiety with R¹⁰X (xiv) when R¹⁰ is other than H. Deprotection of intermediate (xv) and subsequent acylation with a carboxylic acid R¹¹C(O)OH or an activated derivative (xvi) provides the piperidine/1,3-diazinane product according to Formula II.

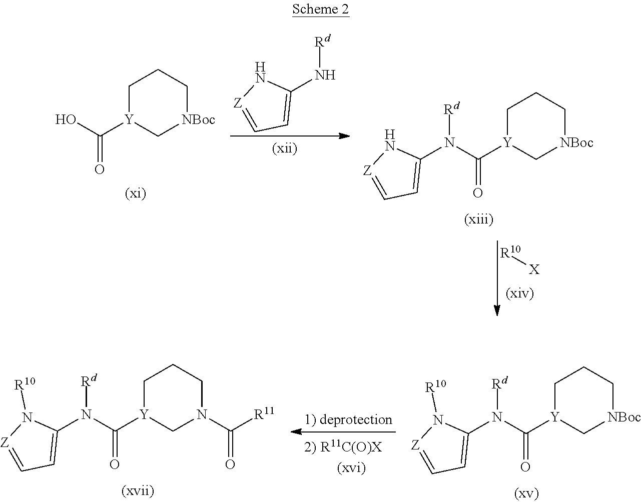

Various coupling agent may be used for the acylation steps in Scheme 1 and Scheme 2 employing carboxylic acids R³C(O)OH and R¹¹C(O)OH. The coupling agent may be, for example, a carbodiimide, a guanidinium salt, a phosphonium salt, or a uronium salt. Examples of carbodiimides include, but are not limited to, N,N′-dicyclo-hexylcarbodiimide (DCC), 1-ethyl-3-(3-dimethylaminopropyl)carbodiimide (EDC), and the like. Examples of phosphonium salts include, but are not limited to, such as (benzotriazol-1-yloxy)tripyrrolidinophosphonium hexafluorophosphate (PyBOP); bromotris(dimethylamino)-phosphonium hexafluorophosphate (BroP); and the like. Examples of guanidinium/uronium salts include, but are not limited to, O-(benzotriazol-1-yl)-N,N,N′,N′-tetramethyluronium hexafluorophosphate (HBTU); 2-(7-aza-1H-benzotriazole-1-yl)-1,1,3,3-tetramethyluronium hexafluorophosphate (HATU); 1-[(1-(cyano-2-ethoxy-2-oxoethylideneaminooxy) dimethylaminomorpholino)] uronium hexafluorophosphate (COMU); and the like. The coupling agent(s) may be employed in conjunction with a base such as a non-nucleophilic base (e.g., triisopropylethylamine, N,N-diisopropylethylamine, or collidine), that are non-reactive or slow to react with protected diazinanes and protected piperidines. Non-limiting examples of protecting groups (e.g., amine protecting groups) include Fmoc, Boc, allyloxycarbonyl (Alloc), benzyloxycarbonyl (Z); 1-(4,4-dimethyl-2,6-dioxocyclohex-1-ylidene)-3-ethyl (Dde); 1-(4,4-dimethyl-2,6-dioxocyclohex-1-ylidene)-3-methylbutyl (ivDde); and 4-methyltrityl (Mtt). Alternatively, acylation can be conducted using activated carboxylic acid derivatives R³C(O)X and R¹¹C(O)X, as depicted in Schemes 1 and 2. The activated carboxylic acid derivatives may be, for example, an acid anhydride, a mixed anhydride, an acid chloride, or an activated ester (e.g., a pentafluorophenyl ester or an N-hydroxysuccinimidyl ester).

Synthetic routes may employ starting materials that are commercially available or those that can be prepared according to known methods, including those described in Fiesers' Reagents for Organic Synthesis Volumes 1-28 (John Wiley & Sons, 2016), by March (Advanced Organic Chemistry 6^{th} Ed. John Wiley & Sons, 2007), and by Larock (Comprehensive Organic Transformations 3^{rd} Ed. John Wiley & Sons, 2018). The synthesis of typical compounds described herein may be accomplished as described in the following examples. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures. Additionally, as will be apparent to those skilled in the art, conventional protecting groups may be advantageous for preventing certain functional groups from undergoing undesired reactions. Suitable protecting groups for various functional groups as well as suitable conditions for protecting and deprotecting particular functional groups are well known in the art. For example, numerous protecting groups are described in Green and Wuts (Protective Groups in Organic Synthesis, 4^{th} Ed. 2007, Wiley-Interscience, New York) and references cited therein.

### C. Pharmaceutical Compositions

In some embodiments, the diazinane or piperidine BET protein inhibitor is administered as a pharmaceutical composition containing at least one pharmaceutically acceptable excipient and the diazinane or piperidine BET protein inhibitor or a pharmaceutically acceptable salt thereof. A diazinane or piperidine BET protein inhibitor may be administered to the subject before administration of one or more additional actives, after administration of one or more additional actives, or concurrently with administration of one or more additional actives. A diazinane or piperidine BET protein inhibitor may be administered in a composition separate from one or more additional actives, or in a composition containing one or more additional active agents. The compositions may be formulated, e.g., for oral administration, intravenous administration, intramuscular administration, intraperitoneal administration, subcutaneous administration, intrathecal administration, intraarterial administration, nasal administration, or rectal administration.

The pharmaceutical compositions can be prepared by any of the methods well known in the art of pharmacy and drug delivery. In general, preparation of the compositions includes the step of bringing the active ingredients into association with a carrier containing one or more accessory ingredients. The pharmaceutical compositions are typically prepared by uniformly and intimately bringing the active ingredients into association with a liquid carrier or a finely divided solid carrier or both, and then, if necessary, shaping the product into the desired formulation. The compositions can be conveniently prepared and/or packaged in unit dosage form.

The pharmaceutical compositions may be in a form suitable for oral use. Suitable compositions for oral administration include, but are not limited to, tablets, troches, lozenges, aqueous or oily suspensions, dispersible powders or granules, emulsions, hard or soft capsules, syrups, elixirs, solutions, buccal patches, oral gels, chewing gums, chewable tablets, effervescent powders, and effervescent tablets. Such compositions can contain one or more agents selected from sweetening agents, flavoring agents, coloring agents, antioxidants, and preserving agents in order to provide pharmaceutically elegant and palatable preparations.

Tablets generally contain the active ingredients in admixture with non-toxic pharmaceutically acceptable excipients, including: inert diluents, such as cellulose, silicon dioxide, aluminum oxide, calcium carbonate, sodium carbonate, glucose, mannitol, sorbitol, lactose, calcium phosphate, and sodium phosphate; granulating and disintegrating agents, such as corn starch and alginic acid; binding agents, such as polyvinylpyrrolidone (PVP), cellulose, polyethylene glycol (PEG), starch, gelatin, and acacia; and lubricating agents such as magnesium stearate, stearic acid, and talc. The tablets can be uncoated or coated, enterically or otherwise, by known techniques to delay disintegration and absorption in the gastrointestinal tract and thereby provide a sustained action over a longer period. For example, a time delay material such as glyceryl monostearate or glyceryl distearate can be employed. Tablets can also be coated with a semi-permeable membrane and optional polymeric osmogents according to known techniques to form osmotic pump compositions for controlled release. Compositions for oral administration can be formulated as hard gelatin capsules wherein the active ingredient is mixed with an inert solid diluent (such as calcium carbonate, calcium phosphate, or kaolin), or as soft gelatin capsules wherein the active ingredients are mixed with water or an oil medium (such as peanut oil, liquid paraffin, or olive oil).

The pharmaceutical compositions can also be in the form of an injectable aqueous or oleaginous solution or suspension. Sterile injectable preparations can be formulated using non-toxic parenterally-acceptable vehicles including water, Ringer's solution, and isotonic sodium chloride solution, and acceptable solvents such as 1,3-butane diol. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

Aqueous suspensions contain the active agents in admixture with excipients suitable for the manufacture of aqueous suspensions. Such excipients include, but are not limited to: suspending agents such as sodium carboxymethylcellulose, methylcellulose, oleagino-propylmethylcellulose, sodium alginate, polyvinyl-pyrrolidone, gum tragacanth and gum acacia; dispersing or wetting agents such as lecithin, polyoxyethylene stearate, and polyethylene sorbitan monooleate; and preservatives such as ethyl, n-propyl, and p-hydroxy benzoate. Oily suspensions can be formulated by suspending the active ingredients in a vegetable oil, for example, arachis oil, olive oil, sesame oil or coconut oil, or in a mineral oil such as liquid paraffin. The oily suspensions can contain a thickening agent, for example beeswax, hard paraffin, or cetyl alcohol. These compositions can be preserved by the addition of an anti-oxidant such as ascorbic acid. Dispersible powders and granules (suitable for preparation of an aqueous suspension by the addition of water) can contain the active ingredients in admixture with a dispersing agent, wetting agent, suspending agent, or combinations thereof.

The pharmaceutical compositions can also be in the form of oil-in-water emulsions. The oily phase can be a vegetable oil, for example olive oil or arachis oil, or a mineral oil, for example liquid paraffin or mixtures of these. Suitable emulsifying agents can be naturally-occurring gums, such as gum acacia or gum tragacanth; naturally-occurring phospholipids, such as soy lecithin; esters or partial esters derived from fatty acids and hexitol anhydrides, such as sorbitan monooleate; and condensation products of said partial esters with ethylene oxide, such as polyoxyethylene sorbitan monooleate.

Transdermal delivery can be accomplished by means of iontophoretic patches and the like. The active ingredients can also be administered in the form of suppositories for rectal administration of the drug. These compositions can be prepared by mixing the active agents with a suitable non-irritating excipient which is solid at ordinary temperatures but liquid at the rectal temperature and will therefore melt in the rectum to release the drug. Such materials include cocoa butter and polyethylene glycols.

### D. Administration of Diazinane and Piperidine BET Protein Inhibitors

Diazinane and piperidine BET protein inhibitors according to the present disclosure, as well as other active agents employed in combination therapy as described herein, can be administered to subject orally, intravenously, intramuscularly, intraperitoneally, subcutaneously, intrathecally, intraarterially, nasally, rectally, or via other routes if indicated. In some embodiments, the diazinane or piperidine BET protein inhibitor is administered orally or via injection. Active agents can be administered at any suitable dose in the methods provided herein. In general, a diazinane or piperidine BET protein inhibitor or other active agent is administered at a dose ranging from about 0.1 milligrams to about 1000 milligrams per kilogram of a subject's body weight (i.e., about 0.1-1000 mg/kg). The dose of the diazinane or piperidine BET protein inhibitor can be, for example, about 0.1-1000 mg/kg, or about 1-500 mg/kg, or about 25-250 mg/kg, or about 50-100 mg/kg. The dose of the diazinane or piperidine BET protein inhibitor can be about 1, 2, 3, 4, 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950 or 1000 mg/kg. In some embodiments, the diazinane or piperidine BET protein inhibitor is administered in an amount ranging from about 0.1 mg/kg/day to about 100 mg/kg/day. In some embodiments, the diazinane or piperidine BET protein inhibitor is administered in an amount ranging from about 0.1 mg/kg/day to about 1.0 mg/kg/day. The dosages can be varied depending upon the requirements of the patient, the severity of the condition, the route of administration, and the particular formulation being administered. The dose administered to a patient should be sufficient to result in a beneficial therapeutic response in the patient. The size of the dose will also be determined by the existence, nature, and extent of any adverse side-effects that accompany the administration of the diazinane or piperidine BET protein inhibitor in a particular patient. The total dosage can be divided and administered in portions over a period of time suitable to treat to the condition.

Animal studies, such as mouse studies, may be useful in determining dosing for humans. For example, an average mouse weighs 0.025 kg. Administering 0.025, 0.05, 0.1 and 0.2 mg of a piperazine BET protein inhibitor per day may therefore correspond to a dose range of 1, 2, 4, and 8 mg of inhibitor/kg/day. If an average human adult is assumed to have a weight of 70 kg, the corresponding human dosage would be 70, 140, 280, and 560 mg of inhibitor per day. Dosages for other active agents (e.g., antiandrogens) may be determined in similar fashion.

A diazinane or piperidine BET protein inhibitor or other active agent can be administered for periods of time which will also vary depending upon the severity of the condition, and the overall condition of the subject to whom the active agent is administered. Administration can be conducted, for example, hourly, every 2 hours, three hours, four hours, six hours, eight hours, or twice daily including every 12 hours, or any intervening interval thereof. Administration can be conducted once daily, or once every 36 hours or 48 hours, once per week, twice per week, or three times per week. Following treatment, a subject can be monitored for changes in their condition and for alleviation of the symptoms of the condition or disease. The dosage of the active agent can either be increased in the event the subject does not respond significantly to a particular dosage level, or the dose can be decreased if an alleviation of symptoms is observed, or if the condition or disease has been remedied, or if unacceptable side effects are seen with a particular dosage.

In some embodiments, the methods of treatment further include administration of one or more additional anti-cancer agents, anti-inflammatory agents, or anti-viral agents. Examples of anti-cancer agents include, but are not limited to, chemotherapeutic agents (e.g., carboplatin, paclitaxel, docetaxel, cabazitaxel, pemetrexed, or the like), tyrosine kinase inhibitors (e.g., erlotinib, crizotinib, osimertinib, or the like), poly (ADP-ribose) polymerase inhibitors (e.g., olaparib, rucaparib, and the like), and immunotherapeutic agents (e.g., pembrolizumab, nivolumab, durvalumab, atezolizumab, or the like). In some embodiments, the methods include administration of radiotherapy, e.g., external beam radiation; intensity modulated radiation therapy (IMRT); brachytherapy (internal or implant radiation therapy); stereotactic body radiation therapy (SBRT)/stereotactic ablative radiotherapy (SABR); stereotactic radiosurgery (SRS); or a combination of such techniques.

Examples of non-steroidal anti-inflammatory agents (NSAIDs) include, but are not limited to, aceclofenac, 5-amino salicylic acid, aspirin, celecoxib, dexibuprofen, diclofenac, diflunisal, etodolac, fenoprofen, flufenamic acid, flurbiprofen, ibuprofen, indomethacin, ketoprofen, ketorolac, loxoprofen, mefenamic acid, nabumetone, naproxen, nimesulide, sulindac, and pharmaceutically acceptable salts thereof. Examples of antiviral agents include, but are not limited to, protease inhibitors (e.g., ritonavir, lopinavir, saquinavir, indinavir, or the like), nucleic acid polymerase inhibitors (e.g., acyclovir, foscarnet, ganciclovir, ribavirin or the like), interferons, and antibodies or other biologics targeting viral binding or entry to host cells.

Examples of agents for combination with BET protein inhibitors in the treatment of cardiovascular disease include, but are not limited to, anticoagulants (e.g., apixaban, dabigatran, edoxaban, heparin, rivaroxaban, warfarin, or the like), antiplatelet agents (e.g., aspirin, clopidogrel, dipyridamole, prasugrel, ticagrelor, or the like), ACE inhibitors (e.g., benazepril, captopril, enalapril, fosinopril, lisinopril, moexipril, perindopril, quinapril, ramipril, trandolapril, or the like), angiotensin II receptor blockers (e.g., azilsartan, candesartan, eprosartan, irbesartan, losartan, olmesartan, telmisartan, valsartan, or the like), beta blockers (e.g., acebutolol, atenolol, betaxolol, bisoprolol/hydrochlorothiazide, bisoprolol, metoprolol, nadolol, propranolol, sotalol, or the like), calcium channel blockers (e.g., amlodipine, diltiazem, felodipine, nifedipine, nimodipine, nisoldipine, verapamil, or the like), cholesterol-lowering agents (e.g., atorvastatin, fluvastatin, lovastatin, pitavastatin, pravastatin, rosuvastatin, simvastatin, niacin, ezetimibe, or the like), Digitalis preparations (e.g., digoxin or the like), diuretics (e.g., acetazolamide, amiloride, bumetanide, chlorothiazide, chlorthalidone, furosemide, hydro-chlorothiazide, indapamide, metalozone, spironolactone, torsemide, or the like), vasodilators (e.g., isosorbide dinitrate, isosorbide mononitrate, hydralazine, nitroglycerin, minoxidil, or the like), and combinations thereof.

In some embodiments, the levels of BET protein activity in a subject may be reduced by from about 25% to about 95% upon treatment of a subject according to the methods of the present disclosure. For example, BET protein activity in the subject may be reduced by from about 35% to about 95%, or from about 40% to about 85%, or from about 40% to about 80% as compared to the corresponding levels of BET protein activity prior to the first administration of the active agent (e.g., 24 hours prior to the first administration of the active agent).

Also provided herein are methods for inhibiting a BET protein. The methods include contacting the BET protein with an effective amount of a diazinane or piperidine compound as described herein. Inhibiting the BET protein generally includes contacting the BET protein with an amount of the diazinane or piperidine compound sufficient to reduce the activity of the BET protein as compared to the BET protein activity in the absence of the diazinane or piperidine compound. For example, contacting the BET protein with the diazinane or piperidine compound can result in from about 1% to about 99% BET protein inhibition (i.e., the activity of the inhibited BET protein ranges from 99% to 1% of the BET protein activity in the absence of the diazinane or piperidine compound). The level of BET protein inhibition can range from about 1% to about 10%, or from about 10% to about 20%, or from about 20% to about 30%, or from about 30% to about 40%, or from about 40% to about 50%, or from about 50% to about 60%, or from about 60% to about 70%, or from about 70% to about 80%, or from about 80% to about 90%, or from about 90% to about 99%. The level of BET protein inhibition can range from about 5% to about 95%, or from about 10% to about 90%, or from about 20% to about 80%, or from about 30% to about 70%, or from about 40% to about 60%. In some embodiments, contacting the BET protein with a diazinane or piperidine compound as described herein will result in complete (i.e., 100%) BET protein inhibition. Inhibiting a BET protein according to the methods of the present disclosure may occur in vitro or in vivo (e.g., following administration of a diazinane or piperidine compound to a subject in the course of treating a condition such as cancer).

### EXAMPLES

### Example 1. Screening of BET Protein Inhibitor Candidates

It was hypothesized that peptidomimetic small molecules that mimic important structural features of LKIRL(399′-403′; SEQ ID NO:4) can bind with, and block the functions of, the ET domain of BET protein. Based on this hypothesis, in silico screening of commercial peptidomimetic libraries was performed. At the outset, a residue sidechain-based pharmacophore model was built based on MLV-IN/ET NMR structure ensemble (PDB Code: 2N₃K) to filter compounds from a library containing 40,460 candidate compounds. The rationale behind this model and pre-filtering criteria was to make the best of the binding mode and features of MLV-IN beta-sheet peptide. The NMR solution structure ensemble provided several insights for the design of non-peptide compounds. Although the polar and charged residue (K400) and R402 of MLV-IN, or D655 and E653 of ET) may not be well defined in NMR solution structure, the backbone and hydrophobic residue are quite consistent among different conformers. Therefore the location of the MLV-IN hydrophobic residue sidechains (e.g., the isobutyl group of L³⁹⁹) and the backbone H-bond donors and acceptors were regarded as pharmacophore features. Any compounds that matched at least four features passed the filtering criteria and were considered as potential ligands, because they have the functionalities that bear similar properties and space distribution as the MLV-IN peptide. The Glide module of the Schrodinger Molecular Modeling Suite was applied for docking studies of pharmacophore hit compounds, and SP (standard precision) protocol was applied for first-stage initial screening, followed by a more accurate re-docking study using XP (extra precision) protocol. LigPrep module was used to prepare the ligands, and 130,950 binding poses in total were generated during the initial SP screening. According to the docking score ranking, the top 2000 poses from the SP docking stage were chosen for next re-docking study using XP protocol, and all the XP docking poses were thereafter refined by Prime MM-GBSA calculation, in which flexibility was given to the residue side chains within 5Å range around the ligand. When the screening was completed successfully, compound prioritization was achieved by multiple score-rankings and visual inspections. For multi-score ranking approach, top 20 compounds ranked by MMGBSA_dG_Bind score, Prime_Energy score, MMGBSA_dG_Bind_HBond score, and MMGBSA_dG_Bind_Lipo score were extracted (80 ligands in total).

Compounds with the top 200 Docking scores, top 100 MMGBSA_dG_Bind scores, top 100 Prime_Energy scores, and top 100 Complex_HBond scores were extracted and assessed using a visual inspection approach. Ligands which showed extended conformations or are able to make the best of the ET backbone H-Bond acceptors and donors were considered as promising candidates. Ligands with aromatic or aliphatic moieties which can utilize the hydrophobic groove around Phe656 of ET were also taken in to account and 40 candidates were chosen using this approach. Collectively, 10 compounds in total were identified as final selections from the library. Poses for each compound were shown in the attached supplementary information. Piperazine compound 10 (N-(3-(4-(2-(4-chlorophenoxy)ethyl)piperazine-1-carbonyl)phenyl)tetrahydrofuran-2-carboxamide) and piperidine compound 2 (1-(3,4-dichlorobenzoyl)-N-(1-isopropyl-1H-pyrazol-5-yl)piperidine-3-carboxamide) were found to be particularly useful as BET protein inhibitors, as described below.

### Example 2. Inhibition of Cancer Cell Growth with Piperazine and Piperidine BET Protein Inhibitors

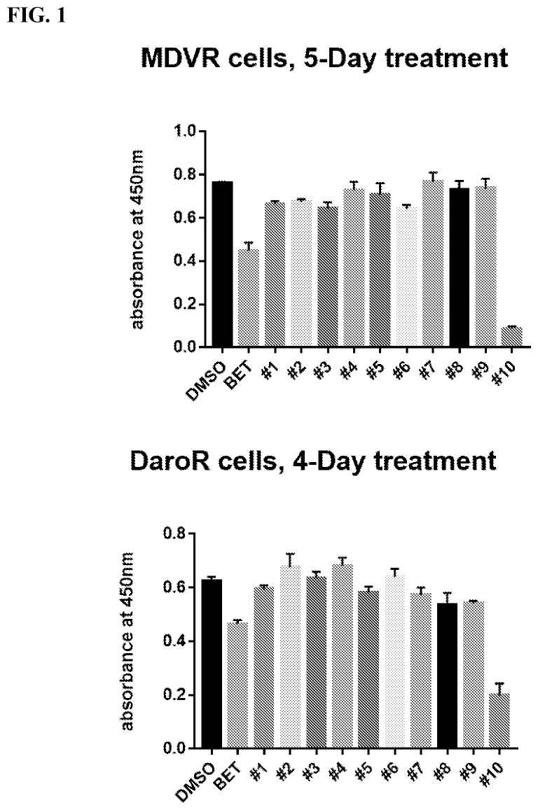

As shown in FIG. 1, piperazine BET protein inhibitor 10 inhibits growth of enzalutamide and darolutamide resistance cells. Enzalutamide resistant C4-2B MDVR cells and darolutamide resistant C4-2B DaroR cells were treated with candidate compounds at 5 μM. BET (I-BET151; (R)-7-(3,5-dimethylisoxazol-4-yl)-8-methoxy-1-(1-(pyridin-2-yl)ethyl)-1,3-dihydro-2H-imidazo[4,5-c]quinolin-2-one; CAS RN 1300031-49-5) was used as the positive control at 5 μM. The cell number was determined. Only piperazine BET protein inhibitor 10 and the BET control inhibited both C4-2B MDVR and C4-2B DaroR cell growth at a concentration of 5 μM. Other compounds from the screen were not observed to effect cell growth under the test conditions.

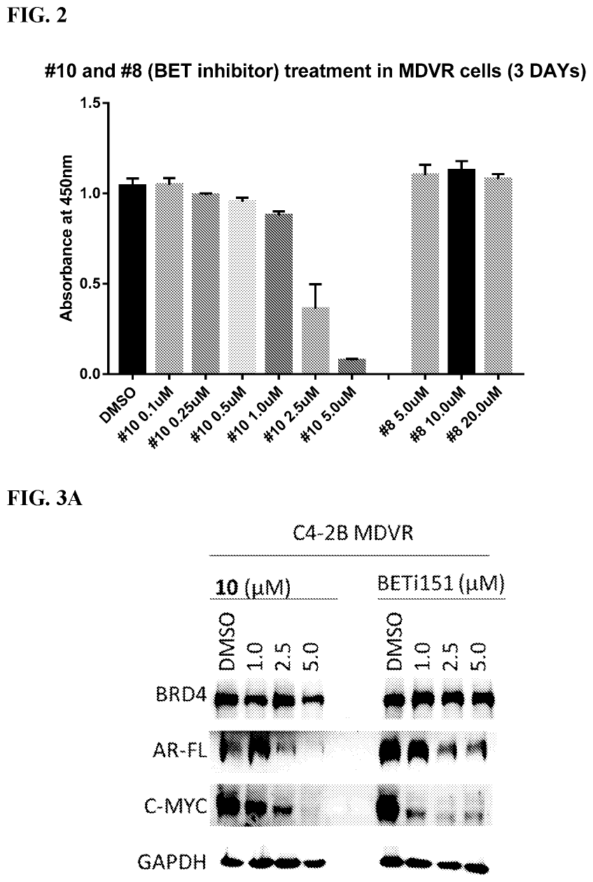

As shown in FIG. 2, piperazine BET protein inhibitor 10 inhibited growth of C4-2B MDVR cells in a dose-dependent manner. C4-2B MDVR cells were treated with increasing doses of piperazine BET protein inhibitor 10 and compound 8 (8-(1-isopropyl-6-methyl-1H-pyrazolo[3,4-b]pyridine-5-carbonyl)-1,3-diazaspiro[4.5]decane-2,4-dione) as indicated. The cell number was determined after 3-day treatment. The results indicated that the IC50 of piperazine BET protein inhibitor 10 is in the range of 1.7-2.1 μM, while no growth inhibition was observed upon treatment with compound 8 at the concentrations used in the experiment.

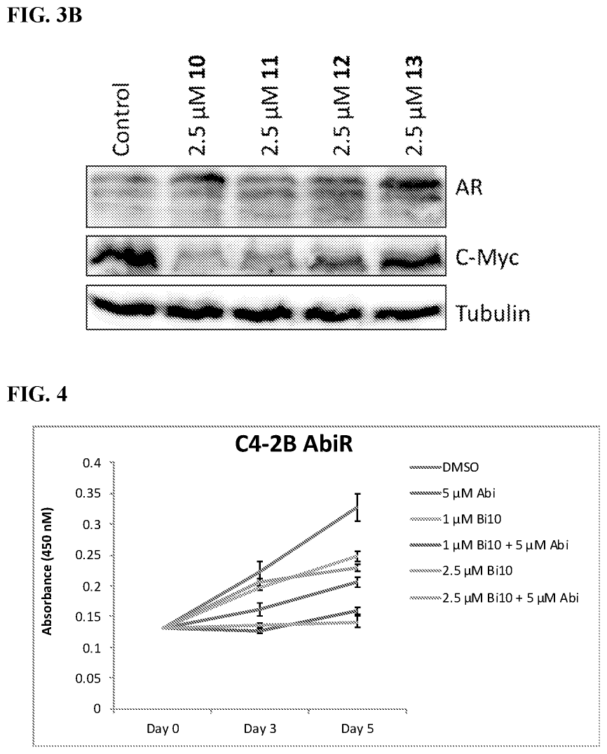

Piperazine BET protein inhibitor 10 inhibited BRD4, AR-FL, and cMYC expression in C4-2B MDVR cells, as shown in FIG. 3. C4-2B MDVR cells were treated with piperazine BET protein inhibitor 10 and the I-BET151 control at increasing doses for 2 days. The cell lysates were subjected to Western blot analysis for BRD4, AR-FL, and cMYC expression. As shown here, both piperazine BET protein inhibitor 10 and I-BET151 inhibited BRD4, AR-FL, and cMYC protein expression.

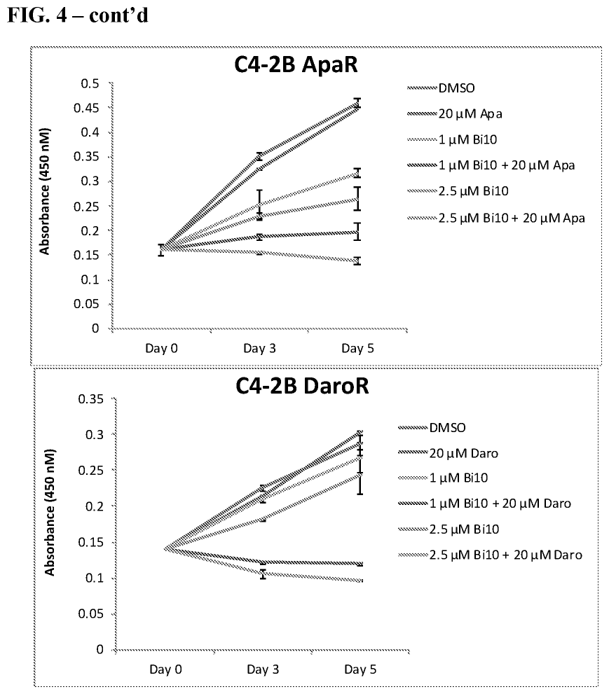

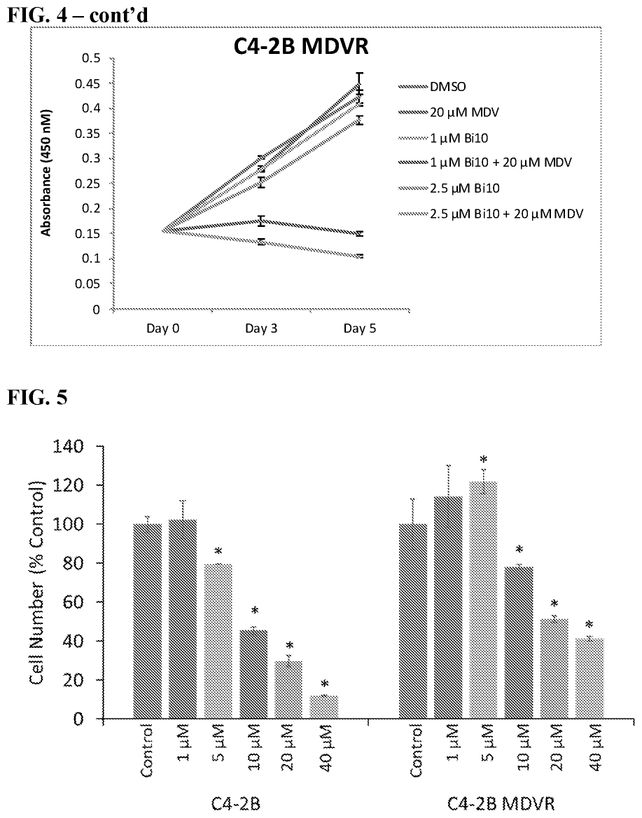

As shown in FIG. 4, piperazine BET protein inhibitor 10 was found to synergize with antiandrogen drugs to inhibit the growth of cancer cells. Piperazine BET protein inhibitor 10 significantly synergized with enzalutamide (MDV) with CDI 0.26, darolutamide (Daro) with CDI 0.38, and apalutamide (Apa) with CDI 0.54, and also synergizes abiraterone (Abi) with CDI 0.98. C4-2B AbiR: abiraterone resistance; C4-2B ApaR: apalutamide resistance; C4-2B DaroR: darolutamide resistance; C4-2B MDVR: enzalutamide resistance. Coefficients of drug interaction (CDI) as determined on day 3 and day 5 are summarized in the following table.

| | 1 μM (10) | 1 μM (10) | 2.5 μM (10) | 2.5 μM (10) |
|---|---|---|---|---|
| Antiandrogen | 3 Day CDI | 5 Day CDI | 3 Day CDI | 5 Day CDI |
| Abi | 1.018586 | 1.362421 | 0.913992 | 0.983485 |
| Apa | 0.798115 | 0.639021 | 0.735802 | 0.538088 |
| Daro | 0.660722 | 0.457359 | 0.611145 | 0.376218 |
| MDV | 0.679134 | 0.346382 | 0.569276 | 0.262753 |

In further experiments, piperidine BET protein inhibitor 2 was found to inhibit the growth of C4-2B and C4-2B MDVR cells in a dose-dependent manner. As shown in FIG. 5, cells were treated with doses of compound 2 (1-40 μM). C4-2B and C4-2B MDVR Cells were plated at 20,000 cells per well in 24-well plates and treated as indicated for 6 days. Total cell number was assessed by Coulter Counter. * indicates significant difference from control for each cell line, p<0.05.

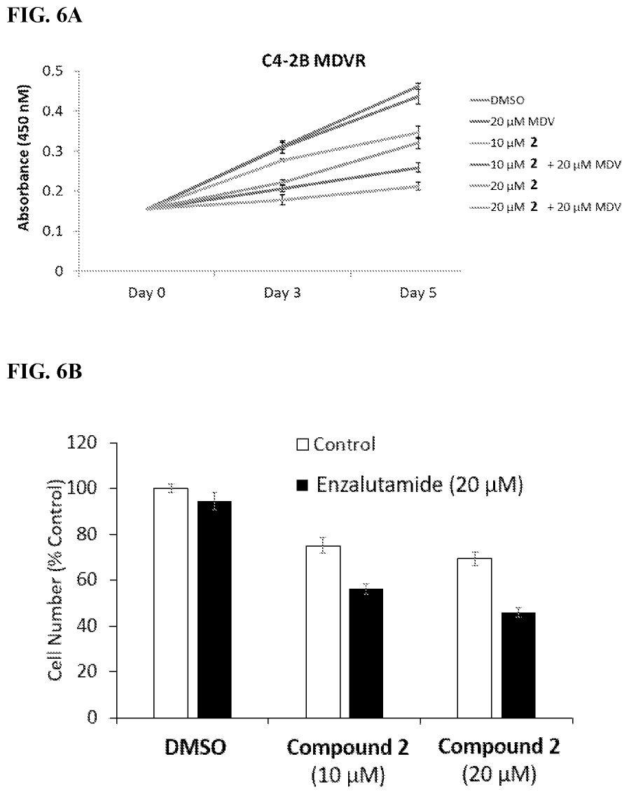

Piperidine BET protein inhibitor 2 was also found to synergize with enzalutamide in inhibiting the growth of enzalutamide-resistant C4-2b MDVR cells, as shown in FIG. 6A and FIG. 6B. CDI values for the experiment shown in FIG. 6A are summarized in the following table.

| Treatment | CDI |
|---|---|
| 3 Day 10 μM 2 | 0.751035 |
| 3 Day 20 μM 2 | 0.810538 |
| 5 Day 10 μM 2 | 0.792049 |
| 5 Day 20 μM 2 | 0.702007 |

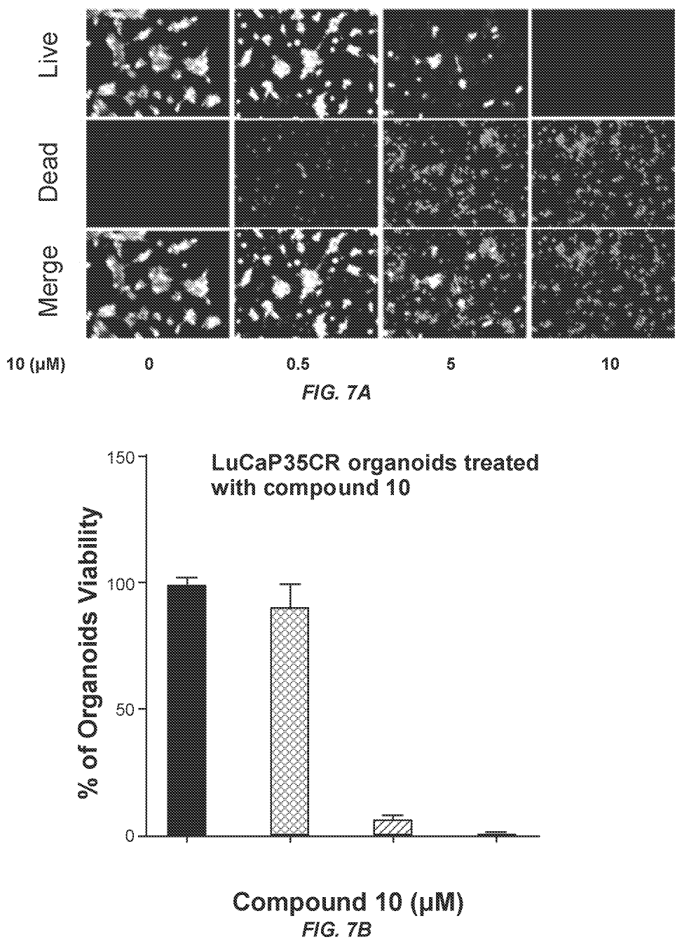

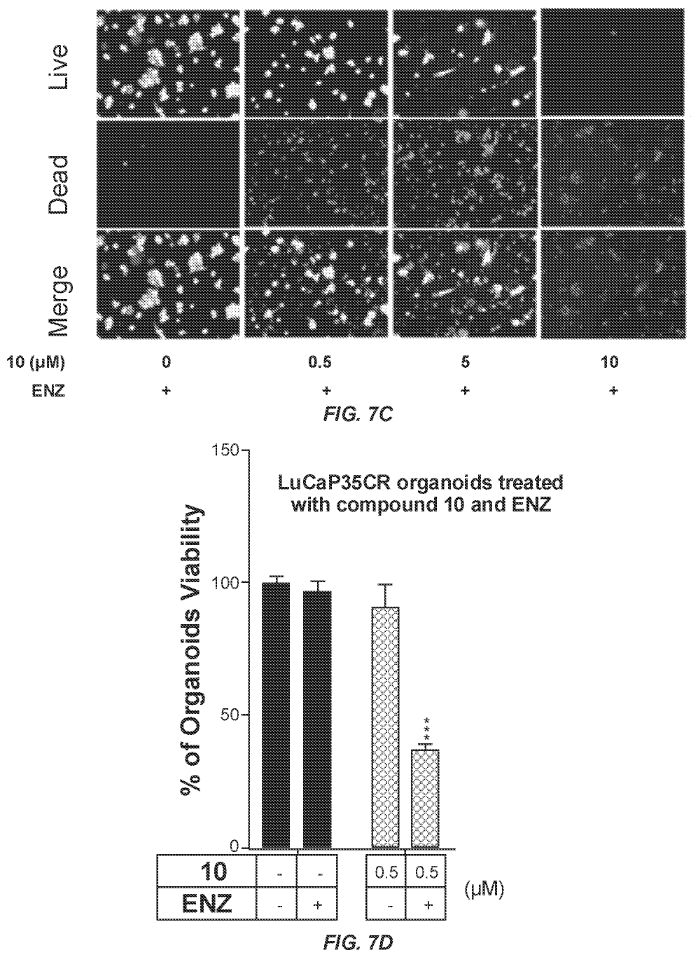

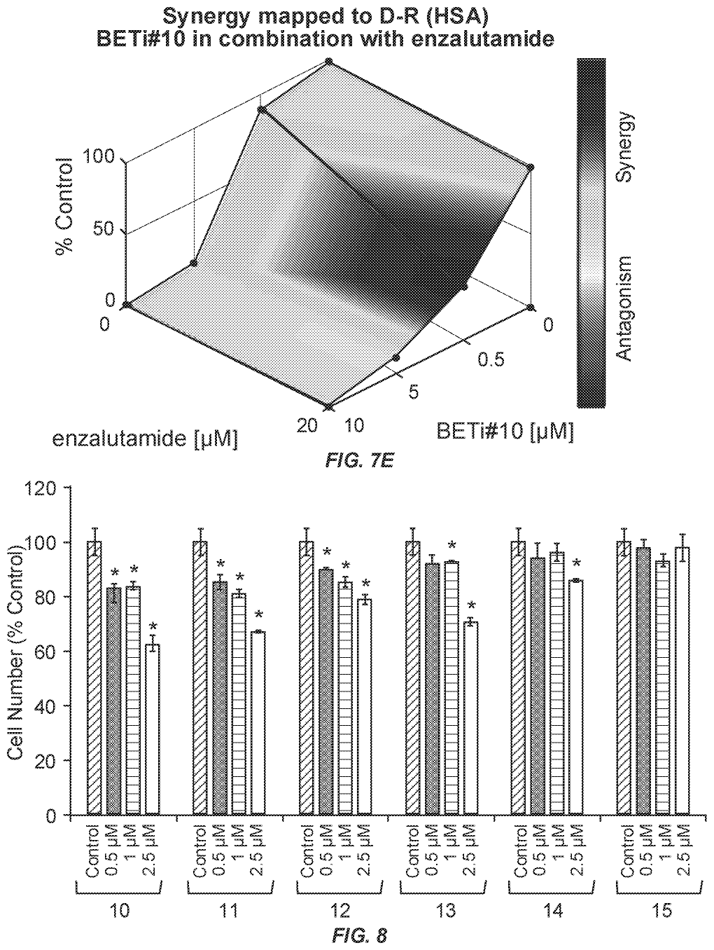

FIG. 7 shows the study of effects of piperazine BET protein inhibitor 10 on LuCaP 35CR organoids. Organoids generated from enzalutamide resistant LuCaP35CR PDX tumors were treated with either piperazine BET protein inhibitor 10 at concentrations as indicated (A) or in combination with enzalutamide 20 μM(C) for 7 days; representative organoids were imaged under the fluorescence microscope after stained by LIVE/DEAD Viability/Cytotoxicity Kit (Invitrogen MP03224). Organoid viability was measured using CellTiter Glo (Promega Catelog#G9681) for piperazine BET protein inhibitor 10 single treatment (B) and for combinational treatment with enzalutamide (D, E). The synergistic effects of piperazine BET protein inhibitor 10 and enzalutamide were demonstrated in using ComBenefit software (Cambridge) (E). Blue indicates synergy while red indicates antagonism between drugs. The coefficient of drug interaction (CDI) is calculated as follows: CDI=AB/(A×B). A is growth inhibition effect of treatment with the BET protein inhibitor. B is growth inhibition effect of enzalutamide treatment. AB is the growth inhibition effect of the combination treatment. The CDI were calculated to determine the synergism of two drugs combinational treatment (CDI<1, =1 or >1 indicates that the drugs are synergistic, additive or antagonistic). The coefficient of drug interaction (CDI) of 20 μM enzalutamide with 0.5 μM BET protein inhibitor combination treatment were 0.97 and 0.91 respectively in LuCaP35CR organoids, evidencing a synergistic effect (CDI=0.4222, <1).

### Example 3. Preparation of BET Protein Inhibitors

Compounds 11-15 were synthesized as outlined in Scheme 3.

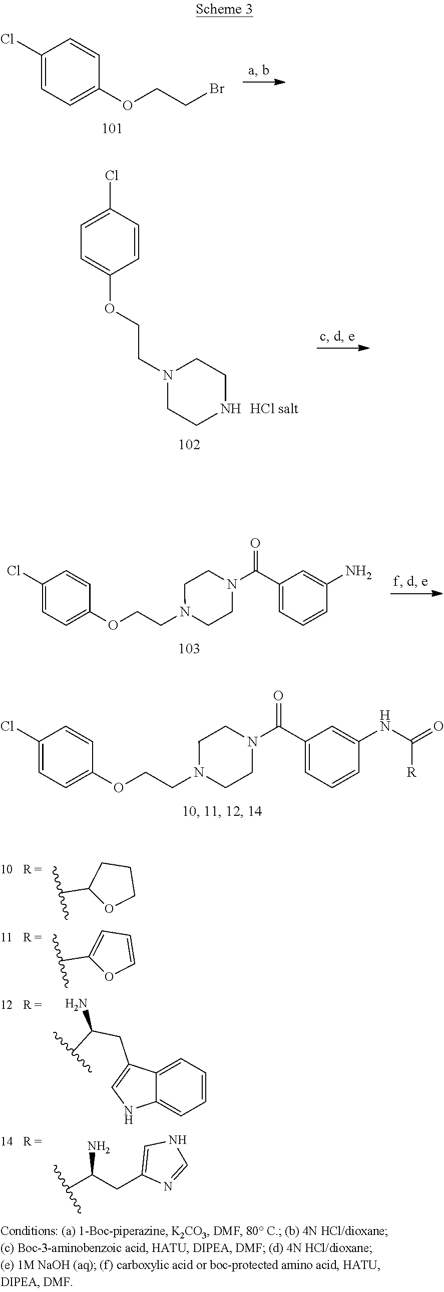

General methods and materials. All solvents were purchased commercially and utilized without further purification. All reagents were obtained from commercial suppliers and used without further purification. Reactions were monitored by thin layer chromatography using silica gel 60 F₂₅₄ aluminum supported plate. Flash column chromatography was performed with CombiFlash NEXTGEN 300+from TELEDYNE ISCO. Melting points were determined using the Uni-Melt apparatus. ¹H and ¹³C NMR spectra were measured on a Bruker ASCENDS400 instrument. High resolution mass spectra were obtained by Electro Spray Ionization (ESI).

1-(2-(4-Chlorophenoxy)ethyl)piperazine hydrochloride (102). To a solution of 1-(2-bromoethoxy)-4-chlorobenzene 101 (936 mg. 4 mmol) in DMF (10 ml) was added tert-butyl piperazine-1-carboxylate (1117 mg, 6 mmol) and K₂CO₃ (1104 mg, 8 mmol), and the mixture was stirred overnight at 80° C. Upon completion, the reaction was quenched with water (20 mL) and extracted with ethyl acetate (40 mL×3). The combined organic layers were washed with brine (25 mL×3), dried over Na₂SO₄ and concentrated under vacuum. The residue was purified by flash column chromatography (EA/HEX) to give 840 mg (62%) of the boc protected intermediate. A mixture of the intermediate (800 mg) and 4N HCl/Dioxane (2 mL) was stirred at room temperature for 2 h. Upon completion, the solvent was removed under vacuum. The solid was suspended in Et₂O, filtered, washed with Et₂O and dried to give the titled compound 102, which was used without further purification. ¹H NMR (400 MHz, Methanol-d₄): δ7.41-7.23 (m, 2H), 7.15-6.98 (m, 2H), 4.48 (t, J=4.8 Hz, 2H), 4.12-3.53 (m, 10H).

(3-Aminophenyl)(4-(2-(4-chlorophenoxy)ethyl)piperazin-1-yl)methanone (103). To a solution of 3-(boc-amino)benzoic acid (237 mg, 1 mmol), HATU (456 mg, 1.2 mmol), and DIPEA (523 μL, 3 mmol) in DMF (5 mL) was added 1-(2-(4-chlorophenoxy)ethyl)piperazine hydrochloride 102 (312 mg, 1 mmol), and the mixture was stirred at room temperature for 8 h. Upon completion, the reaction was quenched with water (10 mL) and extracted with ethyl acetate (40 mL×3). The combined organic layers were washed with brine (25 mL×3), dried over Na₂SO₄ and concentrated under vacuum. The residue was purified by flash column chromatography (EA/HEX) to give 350 mg (76%) of the boc-protected intermediate. A mixture of the intermediate (169 mg, 0.35 mmol) and 4N HCl/Dioxane (2 mL) was stirred at room temperature for 2 h. Upon completion, the solvent was removed under vacuum. The residue was basified with 1N NaOH and extracted with DCM (30 mL×3). The combined organic layers were dried over Na₂SO₄ and concentrated under vacuum. The residue was purified by flash column chromatography (DCM/MeOH) to give 100 mg (80%) of the titled compound 103. ¹H NMR (400 MHz, Methanol-d₄): δ7.31-7.23 (m, 2H), 7.17 (t, J=7.8 Hz, 1H), 6.98-6.90 (m, 2H), 6.79 (ddd, J=8.1, 2.2, 0.8 Hz, 1H), 6.74-6.69 (m, 1H), 6.66 (d, J=7.5 Hz, 1H), 4.14 (t, J=5.4 Hz, 2H), 3.64 (d, J=109.4 Hz, 4H), 2.86 (t, J=5.4 Hz, 2H), 2.63 (d, J=44.5 Hz, 4H).

N-(3-(4-(2-(4-chlorophenoxy)ethyl)piperazine-1-carbonyl)phenyl)tetrahydro-furan-2-carboxamide (10). To a solution of tetrahydrofuran-2-carboxylic acid (70 mg, 0.6 mmol), HATU (228 mg, 0.6 mmol), and DIPEA (261 μL, 1.5 mmol) in DMF (3 mL) was added (3-aminophenyl)(4-(2-(4-chlorophenoxy)ethyl)piperazin-1-yl)methanone 103 (180 mg, 0.5 mmol), and the mixture was stirred at room temperature for 8 h. Upon completion, the reaction was quenched with water (5 mL) and extracted with ethyl acetate (20 mL×3). The combined organic layers were washed with brine (20 mL×3), dried over Na₂SO₄ and concentrated under vacuum. The residue was purified by flash column chromatography (DCM/MeOH) to give 175 mg (77%) of the titled compound 10. ¹H NMR (400 MHz, CDCl₃): δ8.56 (s, 1H), 7.68 (t. J=1.8 Hz, 1H), 7.65-7.54 (m, 1H), 7.37 (t, J=7.9 Hz, 1H), 7.28-7.18 (m, 2H), 7.15 (dt, J=7.5, 1.3 Hz, 1H), 6.89-6.75 (m, 2H), 4.46 (dd, J=8.4, 5.9 Hz, 1H), 4.12-4.00 (m, 3H), 3.95 (q, J=7.0 Hz, 1H), 3.64 (d, J=125.5 Hz, 4H), 2.84 (t, J=5.6 Hz, 2H), 2.60 (d, J=39.5 Hz, 4H), 2.43-2.30 (m, 1H), 2.25-2.08 (m, 1H), 2.06-1.83 (m, 2H). ¹³C NMR (101 MHz, CDCl₃): δ171.56, 169.62, 157.25, 137.45, 136.56, 129.34, 129.28, 125.83, 122.92, 120.82, 118.42, 115.90, 78.59, 69.74, 66.24, 57.00, 53.83, 53.26, 47.67, 42.13, 30.21, 25.61.

N-(3-(4-(2-(4-chlorophenoxy)ethyl)piperazine-1-carbonyl)phenyl)furan-2-carboxamide (11). To a solution of furan-2-carboxylic acid (67 mg, 0.6 mmol), HATU (228 mg, 0.6 mmol), and DIPEA (261 μL, 1.5 mmol) in DMF (3 mL) was added (3-aminophenyl)(4-(2-(4-chlorophenoxy)ethyl)piperazin-1-yl)methanone 103 (180 mg, 0.5 mmol), and the mixture was stirred at room temperature for 8 h. Upon completion, the reaction was quenched with water (5 mL) and extracted with ethyl acetate (20 mL×3). The combined organic layers were washed with brine (20 mL×3), dried over Na₂SO₄ and concentrated under vacuum. The residue was purified by flash column chromatography (DCM/MeOH) to give 197 mg (87%) of the titled compound 11. ¹H NMR (400 MHz, CDCl₃): δ8.28 (s, 1H), 7.75 (s, 1H), 7.68 (d, J=9.2 Hz, 1H), 7.52 (s, 1H), 7.39 (t, J=7.9 Hz, 1H), 7.29-7.13 (m, 4H), 6.83 (d. J=8.9 Hz, 2H), 6.56 (dd, J=3.4, 1.7 Hz, 1H), 4.07 (t, J=5.5 Hz, 2H), 3.65 (d, J=121.3 Hz, 4H), 2.84 (t, J=5.5 Hz, 2H), 2.61 (d, J=32.7 Hz, 4H). ¹³C NMR (101 MHz, CDCl₃): δ169.64, 157.25, 156.21, 147.54, 144.46, 137.57, 136.56, 129.39, 129.34, 125.83, 123.07, 121.20, 118.73, 115.90, 115.56, 112.68, 66.22, 56.99, 53.81, 53.22, 47.65, 42.16.

(S)-2-Amino-N-(3-(4-(2-(4-chlorophenoxy)ethyl)piperazine-1-carbonyl)phenyl)-3-(1H-indol-3-yl)propenamide (12). To a solution of N_{α}-Boc-L-tryptophan (183 mg, 0.6 mmol), HATU (228 mg, 0.6 mmol), and DIPEA (261 μL, 1.5 mmol) in DMF (3 mL) was added (3-aminophenyl)(4-(2-(4-chlorophenoxy)ethyl)piperazin-1-yl)methanone 103 (180 mg, 0.5 mmol), and the mixture was stirred at room temperature for 8 h. Upon completion, the reaction was quenched with water (5 mL) and extracted with ethyl acetate (20 mL×3). The combined organic layers were washed with brine (20 mL×3), dried over Na₂SO₄ and concentrated under vacuum. The residue was purified by flash column chromatography (EA/HEX) to give 290 mg (90%) of the Boc-protected intermediate 13. A mixture of the Boc-protected intermediate (120 mg, 0.18 mmol) and 4N HCl/Dioxane (2 mL) was stirred at room temperature for 2 h. Upon completion, the solvent was removed under vacuum. The residue was basified with 1N NaOH, neutralized with saturated NaHCO₃ (aq) and extracted with DCM (30 mL×3). The combined organic layers were dried over Na₂SO₄ and concentrated under vacuum. The residue was purified by flash column chromatography (DCM/MeOH) to give 57 mg (58%) of the titled compound 12. ¹H NMR (400 MHz, Methanol-d₄): δ7.67-7.57 (m, 2H), 7.52 (ddd, J=8.2, 2.1, 1.0 Hz, 1H), 7.39 (t. J=7.9 Hz, 1H), 7.33 (d, J=8.1 Hz, 1H), 7.30-7.22 (m, 2H), 7.18-7.10 (m, 2H), 7.10-7.02 (m, 1H), 7.01-6.90 (m, 3H), 4.14 (t, J=5.4 Hz, 2H), 3.78 (t, J=6.5 Hz, 3H), 3.47 (s, 2H), 3.37 (s, 1H), 3.30-3.08 (m, 2H), 2.86 (t, J=5.4 Hz, 2H), 2.64 (d, J=45.7 Hz, 4H). ¹³C NMR (101 MHz, Methanol-d₄): δ174.58, 170.56, 157.53, 138.35, 136.75, 135.70, 128.95, 128.87, 127.44, 125.36, 123.39, 122.23, 121.30, 121.05, 118.40, 118.11, 115.69, 110.89, 109.62, 65.60, 56.62, 56.13, 53.30, 52.81, 41.70, 30.89.

(S)-2-Amino-N-(3-(4-(2-(4-chlorophenoxy)ethyl)piperazine-1-carbonyl)phenyl)-3-(1H-imidazol-4-yl)propenamide (14). To a solution of N_{α}-Boc-L-histidine (153 mg, 0.6 mmol), HATU (228 mg, 0.6 mmol), and DIPEA (261 μL, 1.5 mmol) in DMF (3 mL) was added (3-aminophenyl)(4-(2-(4-chlorophenoxy)ethyl)piperazin-1-yl)methanone 103 (180 mg, 0.5 mmol), and the mixture was stirred at 80° C. for 8 h. Upon completion, the reaction was quenched with water (5 mL) and extracted with ethyl acetate (20 mL×3). The combined organic layers were washed with brine (20 mL×3), dried over Na₂SO₄ and concentrated under vacuum. The residue was purified by flash column chromatography (DCM/MeOH) to give 150 mg (50%) of the Boc-protected intermediate 15. A mixture of the Boc-protected intermediate (80 mg, 0.13 mmol) and 4N HCl/Dioxane (2 mL) was stirred at room temperature for 2 h. Upon completion, the solvent was removed under vacuum. The residue was basified with 1N NaOH, neutralized with saturated NaHCO₃ (aq) and extracted with DCM (30 mL×3). The combined organic layers were dried over Na₂SO₄ and concentrated under vacuum. The residue was purified by flash column chromatography (DCM/MeOH) to give 48 mg (74%) of the titled compound 14. ¹H NMR (400 MHz, Methanol-d₄): δ7.76 (s, 1H), 7.68-7.55 (m, 2H), 7.41 (t, J=7.9 Hz, 1H), 7.26 (d, J=8.9 Hz, 2H), 7.15 (d, J=7.6 Hz, 1H), 7.01-6.82 (m, 3H), 4.13 (t, J=5.4 Hz, 2H), 3.89-3.62 (m, 3H), 3.50 (s, 2H), 3.37 (s, 1H), 3.16-3.00 (m, 1H), 2.98-2.80 (m, 3H), 2.64 (d, J=39.1 Hz, 4H). ¹³C NMR (101 MHz, Methanol-d₄): δ173.86, 170.52, 157.52, 138.49, 135.87, 135.00, 133.80, 128.96, 125.33, 122.22, 121.09, 118.19, 116.89, 115.70, 65.60, 56.63, 55.64, 53.33, 52.84, 41.72, 32.45.

Stereoisomeric forms of compound 2 were synthesized as outlined in Scheme 4 for the R-isomer. The S-isomer was prepared according to Scheme 4 using (S)-1-(tert-butoxy-carbonyl)piperidine-3-carboxylic acid in place of (R)-1-(tert-butoxy carbonyl)piperidine-3-carboxylic acid.

Synthesis of ethyl 5-amino-1-isopropyl-1H-pyrazole-4-carboxylate (104): Isopropyl hydrazine hydrochloride (1 eq) was added portion-wise to a solution of ethyl-3-ethoxy2-cyanoacry late (1.1 eq) and N-methyl morpholine (1.1) in EtOH and stirred at room temperature for 1 h. Further, it was stirred at reflux temperature for 16 h and after completion of the reaction half of the EtOH was removed under reduced pressure. The crude compound was purified using EtOAc in hexane as a mobile eluent (White solid, 82% yield): ¹HNMR (400 MHz, CDCl₃) δ7.66 (s, 1H), 5.05 (br, 2H), 4.30-4.18 (m, 3H), 1.48 (d, J=6.4 Hz, 6H), 1.34 (t, J=7.2 Hz, 3H); LC-MS (ESI); [M+H]: 198.10.

Synthesis of 1-isopropyl-1H-pyrazol-5-amine (105); To a solution of ethyl 5-amino-1-isopropyl-1H-pyrazole-4-carboxylate (1.0 eq) in THF:H₂O (3:1), was added LiOH·H₂O (1.5 eq) and stirred overnight at 50° C. The reaction mixture was neutralized with 1M HCl and extracted with EtOAc (3×30 mL), dried over Na₂SO₄, and concentrated on reduced vapor pressure. The residue obtained was stirred in diphenyl ether (30 mL) at 160° C. for 2-3 h. After completion of the reaction (as indicated by TLC), the reaction mixture was cooled and directly loaded over a silica gel column for purification (Transperent liquid, 58% yield): ¹HNMR (400 MHz, CDCl₃) δ10.18 (s, 1H), 7.66 (d, J=2.0 Hz, 1H), 7.28-7.21 (m, 3H), 7.19-7.09 (m, 3H), 6.86 (d, J=8.4 Hz, 1H), 3.86 (s, 2H); LC-MS (ESI); [M+H]: 229.0.

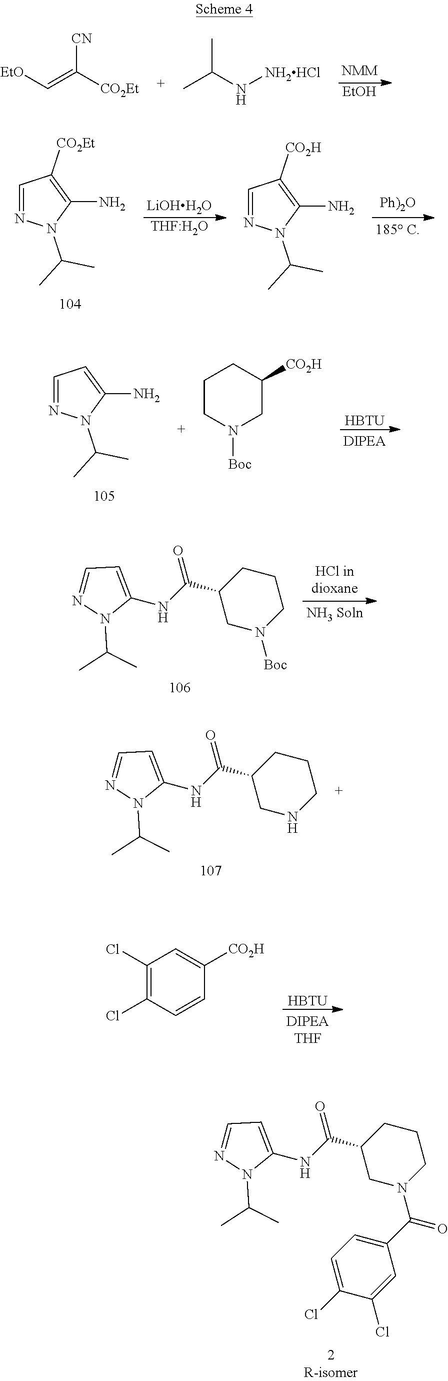

Synthesis of tert-butyl (R)-3-((1-isopropyl-1H-pyrazol-5-yl)carbamoyl)piperidine-1-carboxylate (106): To the solution of (R)-1-(tert-butoxycarbonyl)piperidine-3-carboxylic acid (1.0 eq) in THF, was added HBTU (1.2 Eq), subsequently DIPEA (2.0 Eq), and stirred for 10 min. Then, 1-isopropyl-1H-pyrazol-5-amine (1.0 Eq) was added to the reaction mixture and stirred for overnight at room temperature. The reaction mixture was diluted with EtOAc and H₂O, extracted with EtOAc (3×30 mL). The collective organic layer was washed with 2N NaHCO₃ solution, dried over Na₂SO₄, and concentrated on reduced vapor pressure. The compound was purified by DCM in EtOAC (White solid, 58% yield): ¹HNMR (400 MHz, CDCl₃) δ10.18 (s, 1H), 7.66 (d, J=2.0 Hz, 1H), 7.28-7.21 (m, 3H), 7.19-7.09 (m, 3H), 6.86 (d, J=8.4 Hz, 1H), 3.86 (s, 2H); LC-MS (ESI); [M+H]: 229.06.

Synthesis of (R)-N-(1-isopropyl-1H-pyrazol-5-yl)piperidine-3-carboxamide (107): To a solution of tert-butyl (R)-3-((1-isopropyl-1H-pyrazol-5-yl)carbamoyl)piperidine-1-carboxylate (1.0 eq) in THF, was added HCl in dioxane (5.0 Eq) and stirred for overnight at room temperature. The reaction mixture was diluted with 10% MeOH in DCM and limited of water. The reaction mixture was extracted with 10% MeOH in DCM (3×30 mL) and concentrated on reduced vapor pressure. (White solid, 58% yield): ¹HNMR (400 MHz, CDCl₃) δ10.18 (s, 1H), 7.66 (d, J=2.0 Hz, 1H), 7.28-7.21 (m, 3H), 7.19-7.09 (m, 3H), 6.86 (d, J=8.4 Hz, 1H), 3.86 (s, 2H); LC-MS (ESI); [M+H]: 229.06.

Synthesis of (R)-1-(3,4-dichlorobenzoyl)-N-(1-isopropyl-1H-pyrazol-5-yl)piperidine-3-carboxamide (2): To a solution of 3,4-dichlorobenzoic acid (1.0 eq) in THF, was added HBTU (1.2 Eq), subsequently DIPEA (2.0 Eq), and stirred for 10 min. Then, (R)-N-(1-isopropyl-1H-pyrazol-5-yl)piperidine-3-carboxamide (1.0 Eq) was added to the reaction mixture and stirred overnight at room temperature. The reaction mixture was diluted with EtOAc and H₂O, extracted with EtOAc (3×30 mL). The collective organic layer was washed with 2N NaHCO₃ solution, dried over Na₂SO₄, and concentrated on reduced vapor pressure. The compound was purified by DCM in EtOAc. The compound was precipitated in DCM (2%) in hexanes for extra purification (White solid, 58% yield): ¹HNMR (400 MHz, CDCl₃) δ10.18 (s, 1H), 7.66 (d, J=2.0 Hz, 1H), 7.28-7.21 (m, 3H), 7.19-7.09 (m, 3H), 6.86 (d, J=8.4 Hz, 1H), 3.86 (s, 2H); LC-MS (ESI); [M+H]: 229.06.

### Example 4. Assessment of Piperazine BET Protein Inhibitors In Vitro and In Vivo

As shown in FIG. 8, various piperidine BET protein inhibitors were found to inhibit prostate cancer cell growth. C4-2B MDVR cells were plated at 20,000 cells per well in 24-well plates and treated for 5 days with the indicated compounds. The total cell number was determined and expressed as percentage of control. * indicates a significant difference from control, p<0.05.

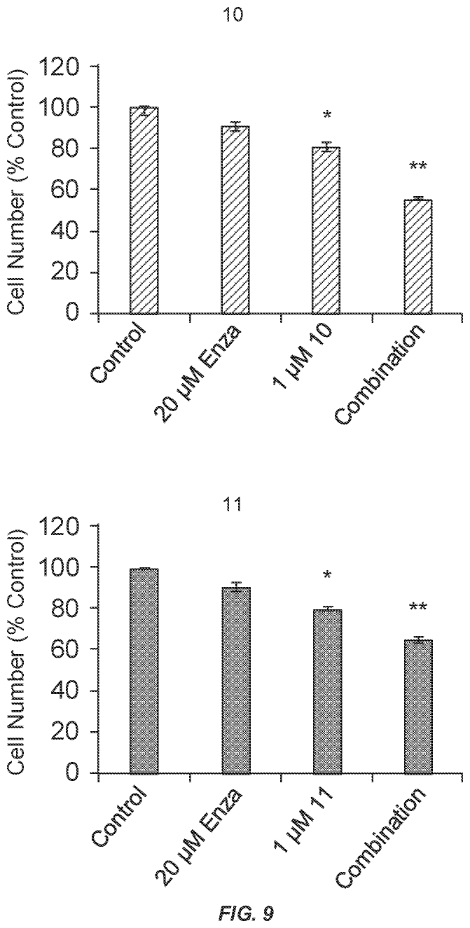

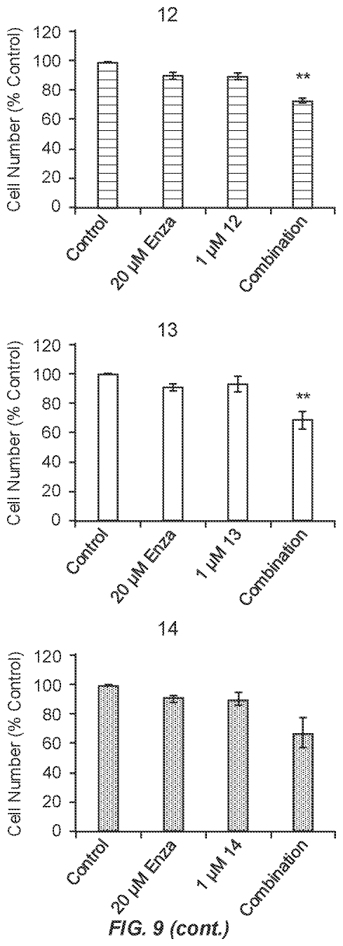

The compounds were also found to synergize with enzalutamide in inhibiting cell growth, as shown in FIG. 9. C4-2B MDVR cells were plated at 20,000 cells per well in 24-well plates and treated for 5 days with the indicated compounds, and then total cell numbers were determined. * indicates significant difference from control and ** indicates significant difference between Enza drug treatment and the combination treatment; p<0.05. CDI values determined for the compounds with enzalutamide are summarized in the following table.

| Compound | CDI with Enzalutamide |
|---|---|
| 10 | 0.76 |
| 11 | 0.90 |
| 12 | 0.90 |
| 13 | 0.81 |
| 14 | 0.82 |

Inhibition of c-myc expression in the cells was by piperazine compounds 10-13 was observed, as shown in FIG. 3B. Analysis of total RNA isolated from cells treated with compound 10 and compound 11 also indicated that the piperazine compounds inhibit the MYC signaling pathway (data not shown).

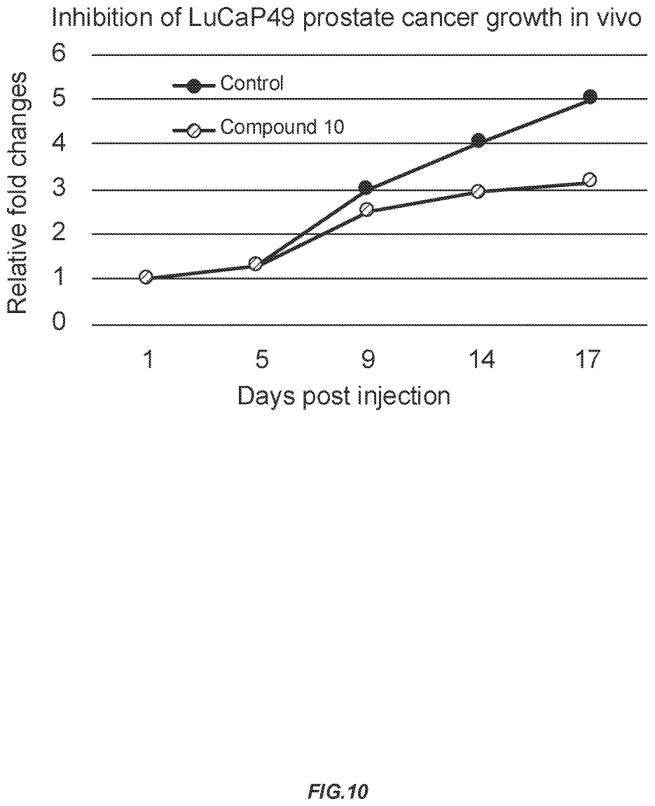

As shown in FIG. 10, piperazine BET protein inhibitor 10 was found to inhibit the growth of prostate cancer tumor growth in mice. Mice bearing tumors from LuCaP49 prostate cancer PDX were treated with BETi-10 (60 mg/kg, i.p.) daily, 5 days/week. Tumor volume were measured and relative fold changes were calculated compared to the day 0 treatment.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

What is claimed is:

1. A bromodomain and extraterminal domain protein (BET protein) inhibitor for use in a method of treating a condition associated with BET protein activity, wherein the BET protein inhibitor is a diazinane BET protein inhibitor or a piperidine BET protein inhibitor, and wherein the method comprises administering an effective amount of the diazinane BET protein inhibitor or the piperidine BET protein inhibitor to a subject in need thereof.

2. The BET protein inhibitor for use of claim 1, wherein the method comprises administering the diazinane BET protein inhibitor to the subject, and wherein the diazinane is a piperazine.

3. The BET protein inhibitor for use of claim 2, having a structure according to Formula I: 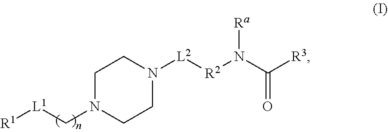
or a pharmaceutically acceptable salt thereof,
wherein:
R¹ is selected from the group consisting of C₆₋₁₄ aryl substituted with one or more R^{1a}, unsubstituted C₆₋₁₄ aryl, and 5- to 10-membered heteroaryl which is optionally substituted with one more R^{1a},
each R^{1a} is independently selected from the group consisting of halogen, —CN, —NO₂, —NHR^{b}, —N₃, —OH, —SH, —SO₃H, C₁₋₈ alkyl, C₁₋₈ alkoxy, C₂₋₈ alkenyl, C₂₋₈ alkynyl, —COOR^{b}, —C(O)NHR^{b}, and —C(O)R^{c},
—L¹— is selected from the group consisting of —O—, —S—, and —NR^{a}—;
—L²— is selected from the group consisting of —C(O)— and —SO₂—;
—R²— is selected from the group consisting of phenylene, pyrrol-diyl, furan-diyl, and thiophen-diyl;
R³ is selected from the group consisting of 5- to 10-membered heterocyclyl, 3- or 4-membered heterocyclyl, C₃₋₈ cycloalkyl, C₆₋₁₄ aryl, and 5- to 10-membered heteroaryl, each of which is optionally substituted with one or more R^{3a},
each R^{3a} is independently selected from the group consisting of halogen, —CN, —NO₂, —NHR^{b}, —N₃, —OH, —SH, —SO₃H, C₁₋₈ alkyl, C₁₋₈ alkoxy, C₂₋₈ alkenyl, C₂₋₈ alkynyl, —COOR^{b}, —C(O)NHR^{b}, and —C(O)R^{c};
alternatively, the grouping —C(O)R³ is an α-aminoacyl moiety;
subscript n is 2, 1, or 3,
each R^{a} and each R^{b} are independently selected from the group consisting of H and C₁₋₄ alkyl; and
each R^{c} is independently C₁₋₄ alkyl.

4. The BET protein inhibitor for use of claim 3, wherein R¹ is phenyl which is substituted with one or more R^{1a}.

5. The BET protein inhibitor for use of claim 4, wherein each R^{1a} is independently halogen.

6. The BET protein inhibitor for use of any one of claims 3-5, wherein —L¹— is —O—.

7. The BET protein inhibitor for use of any one of claims 3-6, wherein —L²— is —C(O)—.

8. The BET protein inhibitor for use of any one of claims 3-7, wherein R² is phen-1,5-diyl.

9. The BET protein inhibitor for use of any one of claims 3-8, wherein R³ is 3- to 10-membered heterocyclyl or 5- to 10-membered heteroaryl.

10. The BET protein inhibitor for use of any one of claims 3-8, wherein the grouping —C(O)R³ is an α-aminoacyl moiety.

11. The BET protein inhibitor for use of claim 10, wherein the α-aminoacyl moiety is selected from the group consisting of histidyl, tryptophanyl, tyrosyl, and phenylalanyl, each of which optionally comprises an α-amino protecting group.

12. The BET protein inhibitor for use of claim 3, which is selected from the group consisting of: 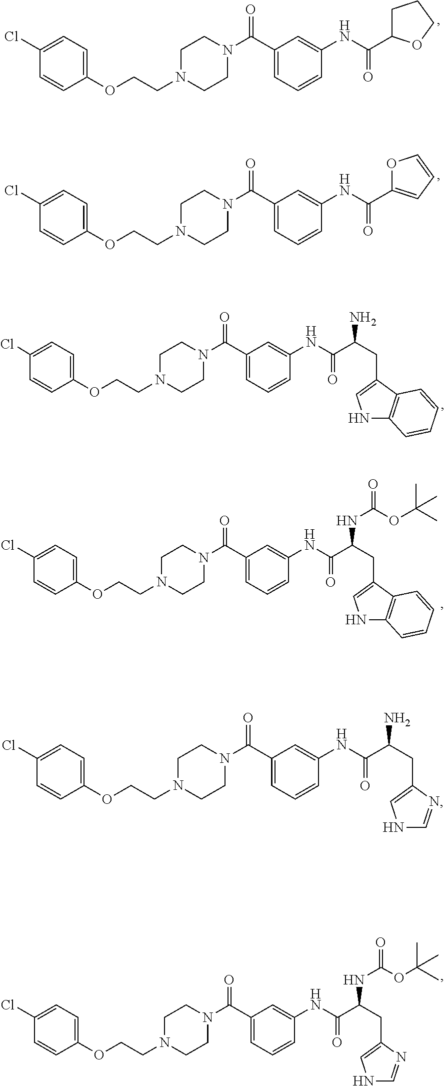 [SMILES: O=C(Nc1cccc(C(=O)N2CCN(CCOc3ccc(Cl)cc3)CC2)c1)C1CCCO1 ; O=C(Nc1cccc(C(=O)N2CCN(CCOc3ccc(Cl)cc3)CC2)c1)c1ccco1 ; N[C@@H](Cc1c[nH]c2ccccc12)C(=O)Nc1cccc(C(=O)N2CCN(CCOc3ccc(Cl)cc3)CC2)c1 ; CC(C)(C)OC(=O)N[C@@H](Cc1c[nH]c2ccccc12)C(=O)Nc1cccc(C(=O)N2CCN(CCOc3ccc(Cl)cc3)CC2)c1 ; N[C@@H](Cc1c[nH]cn1)C(=O)Nc1cccc(C(=O)N2CCN(CCOc3ccc(Cl)cc3)CC2)c1 ; CC(C)(C)OC(=O)N[C@@H](Cc1c[nH]cn1)C(=O)Nc1cccc(C(=O)N2CCN(CCOc3ccc(Cl)cc3)CC2)c1]
and
pharmaceutically acceptable salts thereof.

13. The BET protein inhibitor for use of claim 1, wherein the method comprises administering the diazinane BET protein inhibitor to the subject, wherein the diazinane is a 1,3-diazinane, or wherein the method comprises administering the piperidine BET protein inhibitor to the subject.

14. The BET protein inhibitor for use of claim 13, wherein the BET protein inhibitor is a compound according to Formula II: 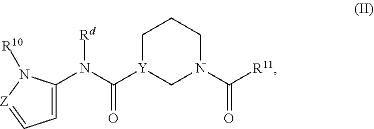
or a pharmaceutically acceptable salt thereof,
wherein:
Y is CH or N;
Z is CH, N, or O;
R¹⁰ is selected from the group consisting of H, C₁₋₈ alkyl, C₃₋₈ cycloalkyl, C₂₋₈ alkenyl, and C₂₋₈ alkynyl, each of which is optionally substituted with one or more R^{10a};
each R^{10a} is independently selected from the group consisting of halogen, —CN, —NO₂, —NHR^{e}, —N₃, —OH, —SH, —SO₃H, C₁₋₈ alkyl, C₁₋₈ alkoxy, C₂₋₈ alkenyl, C₂₋₈ alkynyl, —COOR^{e}, —C(O)NHR^{e}, and —C(O)R^{f};
R¹¹ is selected from the group consisting of C₆₋₁₄ aryl, and 5- to 10-membered heteroaryl, each of which is optionally substituted with one or more R^{11a};
each R^{11a} is independently selected from the group consisting of halogen, —CN, —NO₂, —NHR^{e}, —N₃, —OH, —SH, —SO₃H, C₁₋₈ alkyl, C₁₋₈ alkoxy, C₂₋₈ alkenyl, C₂₋₈ alkynyl, —COOR^{e}, —C(O)NHR^{e}, and —C(O)R^{f};
R^{d} and each R^{e} is selected from the group consisting of H and C₁₋₄ alkyl; and
each R^{f} is independently C₁₋₄ alkyl.

15. The BET protein inhibitor for use of claim 14, wherein R¹⁰ is C₁₋₈ alkyl.

16. The BET protein inhibitor for use of claim 14 or claim 15, wherein R¹¹ is phenyl which is substituted with one or two R^{11a}.

17. The BET protein inhibitor for use of claim 16, wherein each R^{11a} is independently halogen.

18. The BET protein inhibitor for use of any one of claims 14-17, wherein R^{d} is hydrogen.

19. The BET protein inhibitor for use of any one of claims 1-18, wherein the condition associated with BET protein activity is selected from the group consisting of cancer, inflammation, cardiovascular disease, and a viral infection.

20. The BET protein inhibitor for use of claim 19, wherein the cancer is a prostate cancer, an oral cancer, a breast cancer, a lung cancer, or a colon cancer.

21. The BET protein inhibitor for use of claim 19 or claim 20, further comprising administering an antiandrogen drug to the subject.

22. The BET protein inhibitor for use of claim 21, wherein the antiandrogen drug is selected from the group consisting of enzalutamide, apalutamide, bicalutamide, flutamide, nilutamide, darolutamide, and abiraterone.

23. A compound according to Formula I: 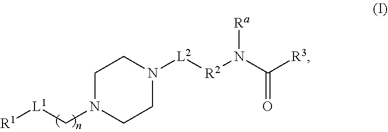
or a pharmaceutically acceptable salt thereof,
wherein:
R¹ is selected from the group consisting of C₆₋₁₄ aryl and 5- to 10-membered heteroaryl, each of which is optionally substituted with one or more R^{1a},
each R^{1a} is independently selected from the group consisting of halogen, —CN, —NO₂, —NHR^{b}, —N₃, —OH, —SH, —SO₃H, C₁₋₈ alkyl, C₁₋₈ alkoxy, C₂₋₈ alkenyl, C₂₋₈ alkynyl, —COOR^{b}, —C(O)NHR^{b}, and —C(O)R^{c},
—L¹— is selected from the group consisting of —O—, —S—, and —NR^{a}—;
—L²— is selected from the group consisting of —C(O)— and —SO₂—;
—R²— is selected from the group consisting of phenylene, pyrrol-diyl, furan-diyl, and thiophen-diyl;
R³ is selected from the group consisting of C₃₋₈ cycloalkyl, 3- to 10-membered heterocyclyl, C₆₋₁₄ aryl, and 5- to 10-membered heteroaryl, each of which is optionally substituted with one or more R^{3a},
each R^{3a} is independently selected from the group consisting of halogen, —CN, —NO₂, —NHR^{b}, —N₃, —OH, —SH, —SO₃H, C₁₋₈ alkyl, C₁₋₈ alkoxy, C₂₋₈ alkenyl, C₂₋₈ alkynyl, —COOR^{b}, —C(O)NHR^{b}, and —C(O)R^{c};
alternatively, the grouping —C(O)R³ is an α-aminoacyl moiety;
subscript n is 1, 2, or 3,
each R^{a} and each R^{b} is independently selected from the group consisting of H and C₁₋₄ alkyl; and
each R^{c} is independently C₁₋₄ alkyl;
provided that:
R³ is substituted with at least one R^{3a} when R¹ is unsubstituted phenyl, chlorophenyl, or methoxyphenyl, —L¹— is —O—, —L²— is —C(O)—, R² is phen-1,5-diyl, and R³ is cyclopropyl, tetrahydrofuranyl, or thiophenyl;
R³ is substituted with at least one R^{3a} when R¹ is unsubstituted phenyl or chlorophenyl, —L¹— is —O— or —S—, —L²— is —C(O)—, R² is thiophen-2,4-diyl, and R³ is cyclopropyl or furanyl;
R¹ is substituted with at least one R^{1a} when R¹ is phenyl, —L¹— is —S—, —L²— is —C(O)—, R² is phen-2,6-diyl, and R³ is fluorophenyl, tetrahydrofuranyl, or cyclopropyl.

24. The compound of claim 23, or a pharmaceutically acceptable salt thereof, wherein R¹ is phenyl which is substituted with one or more R^{1a}.

25. The compound of claim 23 or claim 24, or a pharmaceutically acceptable salt thereof, wherein each R^{1a} is independently halogen.

26. The compound of any one of claims 23-25, or a pharmaceutically acceptable salt thereof, wherein —L¹— is —O—.

27. The compound of any one of claims 23-26, or a pharmaceutically acceptable salt thereof, wherein —L²— is —C(O)—.

28. The compound of any one of claims 23-27, or a pharmaceutically acceptable salt thereof, wherein R² is phen-1,5-diyl.

29. The compound of any one of claims 23-28, or a pharmaceutically acceptable salt thereof, wherein R³ is 5- to 10-membered heteroaryl.

30. The compound of claim 29, or a pharmaceutically acceptable salt thereof, wherein R³ is furanyl.

31. The compound of any one of claims 23-28, or a pharmaceutically acceptable salt thereof, wherein the grouping —C(O)R³ is an α-aminoacyl moiety.

32. The compound of claim 31, or a pharmaceutically acceptable salt thereof, wherein the α-aminoacyl moiety is selected from the group consisting of histidyl, tryptophanyl, tyrosyl, and phenylalanyl, each of which optionally comprises an α-amino protecting group.

33. The compound of claim 32, which is selected from the group consisting of: 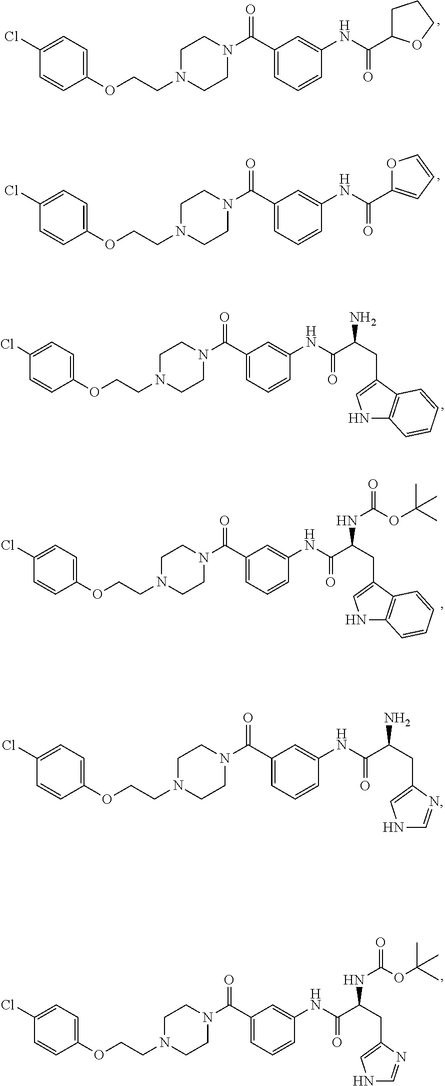 [SMILES: O=C(Nc1cccc(C(=O)N2CCN(CCOc3ccc(Cl)cc3)CC2)c1)C1CCCO1 ; O=C(Nc1cccc(C(=O)N2CCN(CCOc3ccc(Cl)cc3)CC2)c1)c1ccco1 ; N[C@@H](Cc1c[nH]c2ccccc12)C(=O)Nc1cccc(C(=O)N2CCN(CCOc3ccc(Cl)cc3)CC2)c1 ; CC(C)(C)OC(=O)N[C@@H](Cc1c[nH]c2ccccc12)C(=O)Nc1cccc(C(=O)N2CCN(CCOc3ccc(Cl)cc3)CC2)c1 ; N[C@@H](Cc1c[nH]cn1)C(=O)Nc1cccc(C(=O)N2CCN(CCOc3ccc(Cl)cc3)CC2)c1 ; CC(C)(C)OC(=O)N[C@@H](Cc1c[nH]cn1)C(=O)Nc1cccc(C(=O)N2CCN(CCOc3ccc(Cl)cc3)CC2)c1]
and
pharmaceutically acceptable salts thereof.

34. A pharmaceutical composition comprising a compound according to claim any one of claims 23-33, or a pharmaceutically acceptable salt thereof, and one or more pharmaceutically acceptable excipients.

35. A compound according to Formula II: 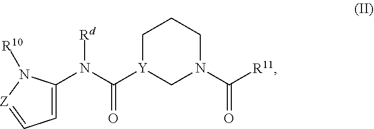
or a pharmaceutically acceptable salt thereof,
wherein:
Y is CH or N;
Z is CH, N, or O;
R¹⁰ is selected from the group consisting of H, C₁₋₈ alkyl, C₃₋₈ cycloalkyl, C₂₋₈ alkenyl, and C₂₋₈ alkynyl, each of which is optionally substituted with one or more R^{10a};
each R^{10a} is independently selected from the group consisting of halogen, —CN, —NO₂, —NHR^{e}, —N₃, —OH, —SH, —SO₃H, C₁₋₈ alkyl, C₁₋₈ alkoxy, C₂₋₈ alkenyl, C₂₋₈ alkynyl, —COOR^{e}, —C(O)NHR^{e}, and —C(O)R^{f};
R¹¹ is selected from the group consisting of C₆₋₁₄ aryl, and 5- to 10-membered heteroaryl, each of which is optionally substituted with one or more R^{11a};
each R^{11a} is independently selected from the group consisting of halogen, —CN, —NO₂, —NHR^{e}, —N₃, —OH, —SH, —SO₃H, C₁₋₈ alkyl, C₁₋₈ alkoxy, C₂₋₈ alkenyl, C₂₋₈ alkynyl, —COOR^{e}, —C(O)NHR^{e}, and —C(O)R^{f};
R^{d} and each R^{e} is selected from the group consisting of H and C₁₋₄ alkyl; and
each R^{f} is independently C₁₋₄ alkyl;
provided that
R¹¹ is substituted with at least one R^{11a} selected from the group consisting of —CN, —NO₂, —NHR^{e}, —N₃, —OH, —SH, —SO₃H, C₁₋₈ alkoxy, C₂₋₈ alkenyl, C₂₋₈ alkynyl, —COOR^{e}, —C(O)NHR^{e}, and —C(O)R^{f} when R¹¹ is phenyl, Y is CH, Z is N, and R¹⁰ is unsubstituted C₁₋₈ alkyl or unsubstituted C₃₋₈ cycloalkyl; and
R¹¹ is substituted with at least one R^{11a} when R¹¹ is furan-2-yl or thiophen-2-yl, Y is CH, Z is N, and R¹⁰ is isopropyl, sec-butyl, cyclopentyl, or cyclohexyl.

36. A pharmaceutical composition comprising a compound according to claim 35, or a pharmaceutically acceptable salt thereof, and one or more pharmaceutically acceptable excipients.
